(19) United States
(12) Patent Application Publication
(10) Pub. No.: US 2016/0229820 A1
(43) Pub. Date: Aug. 11, 2016

(54) PROCESS FOR PREPARING SUBSTITUTED ISATOIC ACID ANHYDRIDE COMPOUNDS AND DERIVATIVES THEREOF

(71) Applicant: BASF SE, Ludwigshafen (DE)

(72) Inventors: Thomas ZIERKE, Boehl-Iggelheim (DE); Jonas HILZENDEGEN, Bellheim (DE); Christopher KORADIN, Neustadt (DE); Karsten KOERBER, Eppelheim (DE); Roland GOETZ, Neulussheim (DE)

(21) Appl. No.: 15/029,907

(22) PCT Filed: Oct. 6, 2014
(86) PCT No.: PCT/EP2014/071285

§ 371 Date: Apr. 15, 2016

(30) Foreign Application Priority Data
Oct. 17, 2013 (EP) 13189104.6
Oct. 18, 2013 (EP) 13189304.2

(51) Int. Cl. C07D 265/26; C07D 401/14; C07D 401/04; C07C 269/04; C07C 271/58

(52) U.S. Cl. CPC C07D 265/26; C07C 269/04; C07C 271/58; C07D 401/04; C07D 401/14

(57) ABSTRACT

The present invention relates to a process for preparing substituted isatoic acid anhydride compounds of the formula (I)

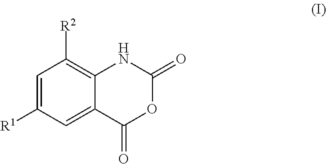

in which
R¹ is Cl, Br, I, or CN; and
R² is CH₃, Cl, Br;
using anthranilic acid derivative compounds of formula (III) and/or carbamate compounds of formula (II)

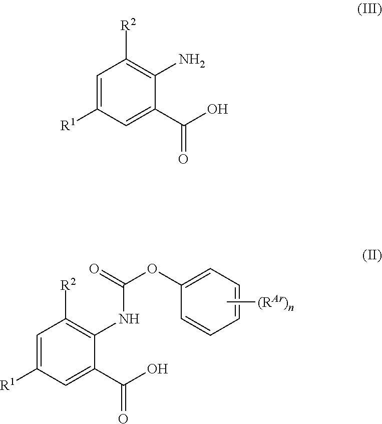

wherein R¹ and R² are as defined above;
R^{Ar} is CH₃, Cl, NO₂ and n is 0, 1, 2, 3, 4 or 5;

The present invention relates also to the compounds of formula (II) and to processes comprising further preceding and/or subsequent reaction steps, leading to anthranilamide pesticides or to precursors for them.

The present invention relates to a process for preparing substituted isatoic acid anhydride compounds and derivatives thereof, in particular anthranilamides. It also relates to the use of these preparing substituted isatoic acid anhydride compounds for preparing anthranilamide derivatives that are useful pesticides. Therefore, substituted isatoic acid anhydride compounds are important precursors for anthranilamide derivatives. Such compounds find use as pesticides, especially as insecticides, which are disclosed, for example, in WO 01/70671, WO 03/015518, WO 03/015519, WO 03/016284, WO 03/016300, WO 03/024222, WO2003/062221, WO2003/027099, WO2004/067528, WO2003/106427, WO 06/000336; WO 06/068669, WO 07/043677, WO2008/126933, WO2008/126858, and WO2008/130021, and in WO2007/006670, WO2013/024009, WO2013/024010, WO2013/024003, WO2013/024004, WO2013/024005, WO2013/024006, WO2013/024169, WO2013/024170, WO2013/024171.

Most processes for preparing anthranilamides, especially anthranilamide pesticides, start from substituted anthranilic acid which is converted to the corresponding substituted isatoic acid anhydride compound.

It is an object of the present invention to provide alternative or improved processes for preparing substituted isatoic acid anhydride compounds and for preparing anthranilamides derived therefrom. These processes should be simple to carry out, require 4 or 3 or less steps overall and be suitable for the industrial scale production. The processes should have good yields and good product purity, and start from readily available starting materials. They should additionally be inexpensive and safe and be based on selective reactions.

The object is achieved by the processes described in detail hereinafter.

In a first aspect, the present invention relates to a process for preparing substituted isatoic acid anhydride compound of the formula (I)

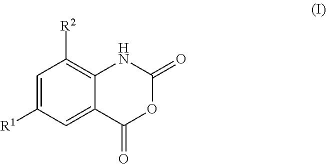

in which
R¹ is Cl, Br, I, or CN; and
R² is CH₃, Cl, Br;
comprising the step (b) of reacting a compound of the formula (II)

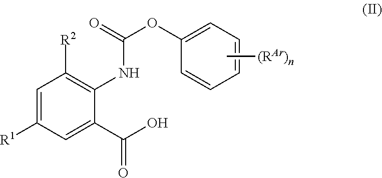

wherein R¹ and R² are as defined above; and
R^{Ar} is CH₃, Cl, NO₂ and n is 0, 1, 2, 3, 4 or 5;
by heating without any further reactants.

Known process routes use phosgen (or diphosgen, triphosgen) for the cyclisation of substituted anthranilic acid to the corresponding substituted isatoic acid anhydride compound. For example, WO2003/015518, WO2003/015519 and WO2033/024222 describe the cyclisation of substituted anthranilic acid to the corresponding substituted isatoic acid anhydride compound, using diphosgen in dioxane. JP2008280344 describes such a cyclisation with triphosgen in tetrahydrofurane. Due to its toxicity, phosgen has some restrictions in its handling and transportation. It is therefore preferably used in sealed systems, where it is produced and reacted. Similar restrictions apply for diphosgen and triphosgen.

WO2008/010897 describes the synthesis of isatoic anhydride synthesis via alkylcarbamates of anthranilic acid derivatives with phosphor tribromide (PBr₃). This has the advantage that phosgen can be avoided. However, PBr₃ is an expensive reactant which is highly corrosive. This results in high costs which prevent a cost-efficient synthesis of isatoic anhydride compounds of formula (I), especially on an industrial scale of production.

WO2008010897 is considered as closest state of the art.

The present invention differs from WO200810897 by the fact that no reactant (PBr₃ or base) is needed.

The technical effect achieved by this difference is that less chemicals are needed while the same result is achieved.

The technical problem was therefore to find a process with less chemicals, while the same result is achieved.

This problem is solved by the present invention. There was no hint in the state of the art to omit the reactant chemicals while keeping the mild reaction conditions.

U.S. Pat. No. 3,238,201, cited in a review by Coppola (“The Chemistry of Isatoic Anhydride”, Synthesis, Vol. 7, 1 Jan. 1980, p.505-536, especially p. 506) refers to the preparation of N-Aryl substituted isatoic acid anhydrides simply by prolonged heating of alkoxy carbamate precursors with or without using a solvent at temperatures between 140° C. (solvent: xylene) and 170° C. (without solvent). The same application proposes the use of thionyl chloride as a condensation auxiliary to overcome the harsh reaction conditions of the only thermal cyclisation approach.

The preparation of N—H isatoic acid anhydrides of the present invention by thermal cyclisation of carbamate precursors is not yet disclosed. Also the more recent teaching of WO2008/010897 does not take into consideration this solution for preparing N—H isatoic acid anhydrides for the complex anthranilamide pesticides. This clearly shows, that a person skilled in the art would not have taken into account this rather special aspect of the Coppola review. It becomes obvious, that the thermal cyclisation is not a general method of preparing N—H-isatoic anhydrides due to various side reactions, which might occur on the stage of the precursors.

For this reasons providing specific precursors, ie. the phenyl carbamates of formula (II), which allow for a thermal cyclisation to the desired N—H-isatoic anhydrides of formula (i) in high yield and with high purity, is based on an inventive step.

An object of the present invention was therefore to provide an economical process for the preparation of the substituted isatoic acid anhydride compounds of formula (I).

A further advantage of the processes according to the invention is that the reagents to be used are safe and inexpensive, which is favourable in view of costs and safety aspects. The reactants are cheap and readily available or can be easily manufactured. Due to these properties, the processes are therefore suitable for production on an industrial scale, which is a further advantage.

The reactant compound of formula (II) can be obtained by reaction of substituted anthranilic acid with arylchloroformate. Therefore, in a further aspect, the invention also relates to a process for preparing a compound of formula (II)

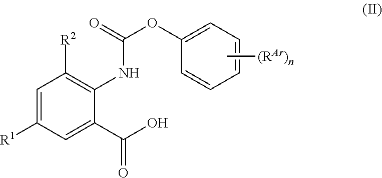

in which
R¹ is Cl, Br, I, or CN; and
R² is CH₃, Cl, Br;
R^{Ar} is CH₃, Cl, NO₂ and n is 0, 1, 2, 3, 4 or 5;
by reacting in a step (a) anthranilic acid derivative compounds of formula (III)

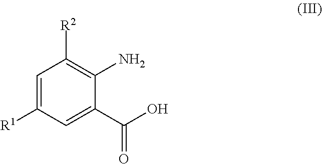

in which R¹ and R² are as defined above,
with a chloroformate compound of formula (IV)

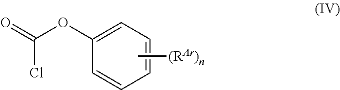

wherein R^{Ar} and n are as defined above;
in the presence of a solvent and without any further reactants.

The term <without any further reactants> is meant to be understood as the absence of further chemical compounds involved in the reaction. Such further chemicals, which are not present in this invention, are e.g. acids or bases, agents for oxidation or reduction. In the present case, this means especially the absence of a base or PBr₃. This does not exclude the presence of solvent and other inerts.

In a further embodiment, the invention relates to a process for preparing a substituted isatoic acid anhydride compound of the formula (I)

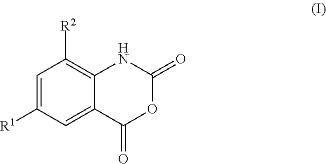

in which
R¹ is Cl, Br, I, or CN; and
R² is CH₃, Cl, Br;

wherein in a step (a) the compound of formula (II)

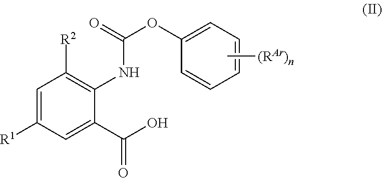

in which
R¹ is Cl, Br, I, or CN; and
R² is CH₃, Cl, Br; and
R^{Ar} is CH₃, Cl, NO₂ and n is 0, 1, 2, 3, 4 or 5;
is prepared by reacting an anthranilic acid derivative compound of formula (III)

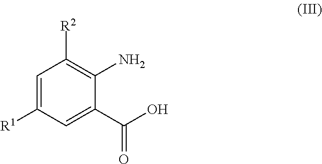

in which R¹ and R² are as defined above,
with a chloroformate compound of formula (IV)

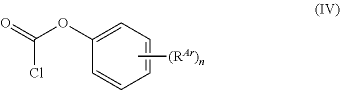

in which R^{Ar} and n are as defined above;
in the presence of a solvent and without any further reactants;
and

comprising a step (b) of reacting a compound of the formula (II)

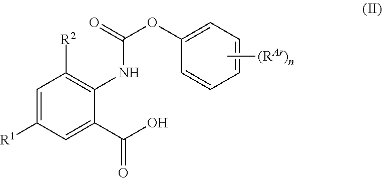

wherein R¹ and R² are as defined above; and
R^{Ar} is CH₃, Cl, NO₂ and n is 0, 1, 2, 3, 4 or 5;

by heating without any further reactants.

This process yields compounds of formula (I) as described above.

In a further embodiment, the invention relates to a process for preparing a substituted isatoic acid anhydride compound of the formula (I)

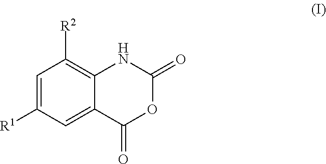

in which
R¹ is Cl, Br, I, or CN; and
R² is CH₃, Cl, Br;
wherein an anthranilic acid derivative compound of formula (III)

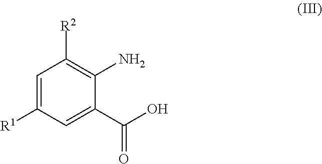

in which R¹ and R² are as defined above,
is reacted with a chloroformate compound of formula (IV)

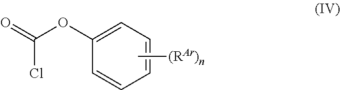

in which R^{Ar} and n are as defined above;
in the presence of a solvent and without any further reactants.

This may be considered as a step-wise reaction or a one-pot reaction.

The conversion is carried out by adding a compound of formula (III) and a compound of formula (IV), preferably in a solvent, and heating the mixture. The heating is mostly for several hours, and mostly under reflux.

As an intermediate compound, phenylcarbamates of formula (II) are formed. The compounds of formula (II) react and form compounds of formula (I).

In one embodiment, the reaction is carried out in a solvent. In one embodiment, the reaction step (b) is carried out in a solvent.

Therefore the invention relates to a process according to the invention, in which the solvent is selected from aromatic hydrocarbon solvents or polar aprotic solvents. Polar aprotic solvents are e.g. acetonitrile, tetrahydrofurane.

The invention furthermore relates to a process according to the invention, in which the solvent is selected from toluene, ethylbenzene, o-xylene, m-xylene, p-xylene, chlorbenzene, or a mixture thereof, preferably toluene.

The invention furthermore relates to a process according to the invention, in which the solvent is selected from acetonitrile, n-butyl acetate and tetrahydrofurane, preferably n-butyl acetate.

The invention furthermore relates to a process according to the invention, in which the solvent is selected from acetonitrile, and tetrahydrofurane.

In another embodiment, the reaction is carried out in an organic solvent which is selected from toluene, ethylbenzene, o-xylene, m-xylene, p-xylene, chlorbenzene, hexane, cyclohexane, methylcyclohexane, or a mixture thereof.

The temperature at which the reaction is carried out (reaction temperature) may be varied in broad ranges, which the person skilled in the art knows. If a solvent is used, the reaction temperature often depends from the reflux temperature of the solvent to be used. In one embodiment, the reaction is carried out at a temperature between 15 to 150° C., or 20 to 150° C., or 20 to 120° C., or 25 to 120° C., or 30 to 120° C., or 40 to 120° C., or 50 to 120° C., or 60 to 120° C., or 70 to 120° C.

In one embodiment, the reaction of step (a) is carried out at room temperature, i.e. between 15-30° C., more preferably between 20-25° C. In one embodiment of the invention, the reaction of step (b) is carried out at a temperature between 60 and 120° C.

The duration time of the reaction varies depending on the amount of acid and depending on the reaction temperature. The end of the reaction can be monitored by methods known to the person skilled in the art, e.g. thin layer chromatography, HPLC. In one embodiment, the reaction is carried out under heating to reflux for up to 20 hours.

The chloroformate compound of formula (IV) is employed in at least equimolar amounts regarding compound of formula (III), preferably in slight excess, e.g. in an excess of 0.1 to 0.5 molar equivalents. The amount of compound (IV) can be varied in certain ranges. It may e.g. be varied from 1 to 10 equivalents [=“eq”, in relation to the compound (III)], or from 1.0 to 5 eq, or from 1.0 to 3 eq, or from 1.0 to 3 eq, or from 1.1 to 2 eq.

“Excess” ratio means that the number of equivalents is bigger than 1, e.g. 1.05 eq, 1.1 eq, 1.15 eq, 1.2 eq, 1.25 eq, 1.3 eq, 1.35 eq, 1.4 eq, 1.45 eq, 1.5 eq, 1.6 eq, 1.7 eq, 1.75 eq, 1.8 eq, 1.9 eq. In one embodiment, the number of equivalents is smaller than 0.5.

The chloroformate compounds of formula (V) may be purchased (e.g. phenylchloroformate from TCI Fine Chemicals, Saltigo etc.) or may be synthesized according to procedures known in the literature, e.g. CS 202458 B1, DE 4137557.

In one embodiment, the chloroformate compound of formula (IV) is phenylchloroformate, i.e. n is 0 in the compound of formula (IV).

Therefore, the present invention relates to processes as described herein, wherein the compound of formula (IV) is phenylchloroformate. In particular, the invention relates to the conversion of compounds of formula (III) with phenylchloroformate to yield a compound of formula (II) [step a]. Also in particular, the invention relates to the conversion of compounds of formula (II) to compounds of formula (I), wherein n is 0, i.e. no R^{Ar} is present [step b]. Also in particular, the invention relates to a combination of steps a and b, wherein n is 0 i.e. no R^{Ar} is present. Also in particular, the invention relates to a conversion of compounds of formula (III) to compounds of formula (I).

The person skilled in the art knows the best work-up of the reaction mixture after the end of the reaction. In one embodiment, the work-up usually isolation of the product by filtration, and optionally washing with solvent, further optionally drying of the product if necessary.

The compound of formula (I) may be employed as crude product in the next reaction step towards the insecticidal compounds described in the beginning. Alternatively, the compound of formula (I) may be purified by methods known to the person skilled in the art and may be employed as a pure compound in the next reaction step towards the insecticidal compounds described in the beginning.

Only two compounds of formula (II) as used in the processes according to the invention are already known per se: 3,5-dibromo-2-(phenoxycarbonylamino)benzoic acid, and 3,5-dichloro-2-(phenoxycarbonylamino)benzoic acid (Aurora Building Blocks, Accession numbers in Chemcats: 0156705781, respectively 0144532873). The use of these compounds in the processes according to the invention has not been described so far.

In one embodiment, the invention relates to a compound of formula (II)

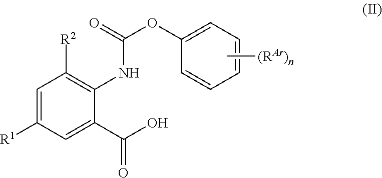

in which
R¹ is Cl, Br, I, or CN; and
R² is CH₃, Cl, Br; and
R^{Ar} is CH₃, Cl, NO₂ and n is 0, 1, 2, 3, 4 or 5.

In one sub-embodiment of the compounds of formula (II), the substituents R¹ and R² are not both Cl or not both Br at the same time.

In another embodiment, the invention relates to a compound of formula (II)

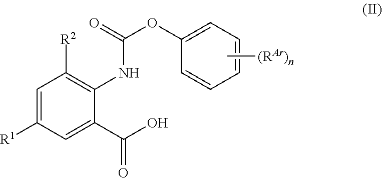

in which
R¹ is Cl, Br, I, or CN; and
R² is CH₃, and
R^{Ar} is CH₃, Cl, NO₂ and n is 0, 1, 2, 3, 4 or 5.

In a further embodiment, the invention relates to compounds of formula (II), wherein n is 0, i.e. compounds of formula (II-Ph):

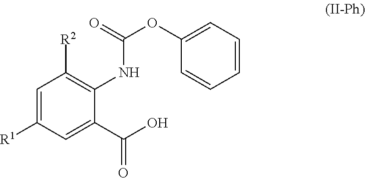

in which
R¹ is Cl, Br, I, or CN; and
R² is CH₃, Cl, Br;
with the proviso that the substituents R¹ and R² are not both Cl or not both Br at the same time.

In one sub-embodiment, the invention relates to compounds of formula (II-Ph) as described above, wherein R² is CH₃.

In a further embodiment, the invention relates to a compound selected from compounds (II-Ar1a), (II-Ar1b) and (II-1c):

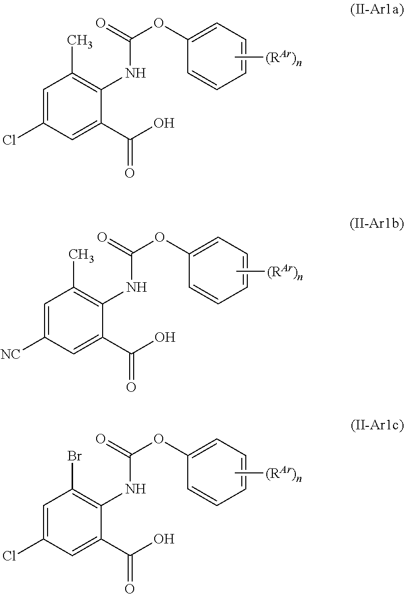

wherein R^{Ar} is CH₃, Cl, NO₂ and n is 1, 2, 3, 4 or 5.

In a further embodiment, the invention relates to a compound of formula (II-1a), which is a compound of formula (II) as described herein, in which R¹ is Cl and R² is CH₃:

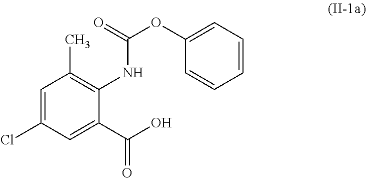 [SMILES: Cc1cc(Cl)cc(C(=O)O)c1NC(=O)Oc1ccccc1]

In a further embodiment, the invention relates to a compound of formula (II-1b), which is a compound of formula (II) as described herein, in which R¹ is CN and R² is CH₃:

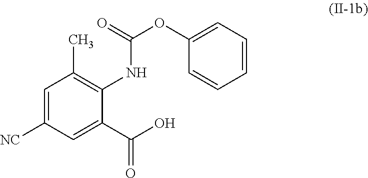 [SMILES: Cc1cc(C#N)cc(C(=O)O)c1NC(=O)Oc1ccccc1]

In a further embodiment, the invention relates to a compound of formula (II-1c), which is a compound of formula (II) as described herein, in which R¹ is Cl and R² is Br:

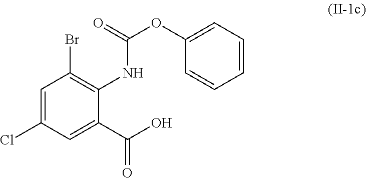 [SMILES: O=C(Nc1c(Br)cc(Cl)cc1C(=O)O)Oc1ccccc1]

As said above, the compounds of formula (I) are useful precursors in the synthesis of anthranilamide pesticides. Therefore, the present invention relates to the use of a compound of formula (II),

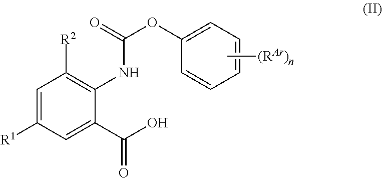

in which
R¹ is Cl, Br, I, or CN; and
R² is CH₃, Cl, Br; and
R^{Ar} is CH₃, Cl, NO₂ and n is 0, 1, 2, 3, 4 or 5;

especially of formula (II-1a) as described above, in the synthesis of anthranilamide pesticides of formula (A):

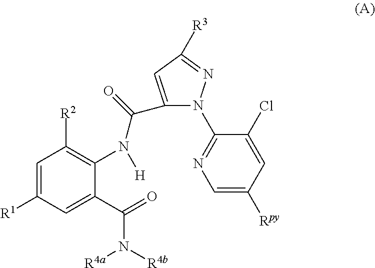

in which
R¹ is Cl, Br, I, or CN;
R² is CH₃, Cl, Br;
R³ is Cl, Br, I, CN, CF₃, CHF₂, OCH₂F or a residue of formula T:

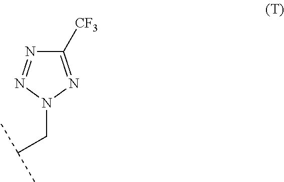

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

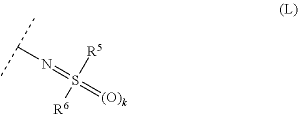

wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
   and
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy;
   phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or
   R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
   two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
n is 0, 1 or 2.

WO2008/010897 discloses compounds which are analogous to the compounds of formula (II) of the present invention, but which have a alkoxy or benzyloxy group instead of the phenyloxy group in the compounds of formula (II) of the present invention. The phenyloxy substituted precursors of formula (II) represent the advantage that these precursors can be cyclized in situ by simple heating of the reaction mixture without adding any auxiliary chemicals.

Furthermore, the present invention relates to the use of a compound of formula (II), especially of formula (II-1a), in the synthesis of anthranilamide pesticides of formula (A-0):

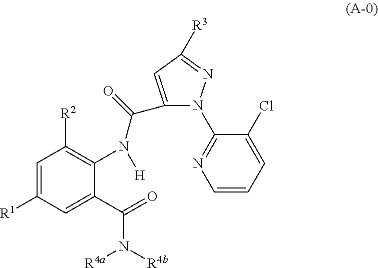

in which
R¹ is Cl, Br, I, or CN;
R² is CH₃, Cl, Br;
R³ is Cl, Br, I, CN, CF₃ or CHF₂;
R^{4a} and R^{4b} are independently selected from hydrogen, C₁-C₄-alkyl,
or
R^{4a} and R^{4b} together form a group (L)

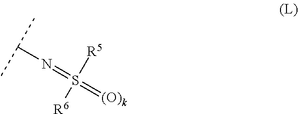

wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
   and
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
   two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
   and especially wherein R^{a}, R^{b}, R^{c}, R^{d}, R^{f}, k and n are as defined above.

Furthermore, the present invention relates to the use of a compound of formula (II), especially of formula (II-1a), in the synthesis of anthranilamide pesticides of formula (A), in which
in which
R¹ is Cl, Br, I, or CN;
R² is CH₃, Cl, Br;
R³ is Cl, Br, I, CN, CF₃, CHF₂, OCH₂F, or a residue of formula T:

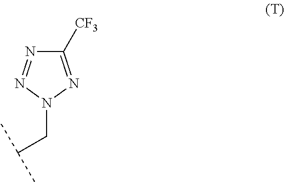

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

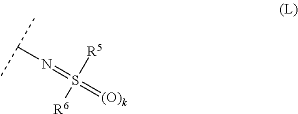

wherein
R⁵, R⁶ are selected independently of one another from the group consisting of hydrogen, C₁-C₄-alkyl, C₃-C₈-cycloalkyl, or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring,
k is 0 or 1.

This refers especially to the use of compounds of formula (II-1a), in the synthesis of anthranilamide pesticides of formula (A) as defined above, in which R¹ is Cl and R² is CH₃.

Furthermore, in one embodiment, the present invention relates to the use of a compound of formula (II), in the synthesis of anthranilamide pesticides of formula (A), in which
R¹ is Cl, Br, I, or CN;
R² is CH₃, Cl, Br;
R³ is Cl, Br, I, CN, CF₃, CHF₂, OCH₂F, or a residue of formula T:

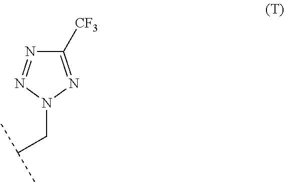

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

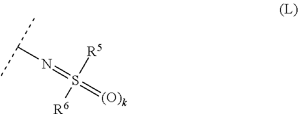

wherein
R⁵, R⁶ are selected independently of one another from the group consisting of hydrogen, C₁-C₄-alkyl and C₃-C₈-cycloalkyl. k is 0 or 1.

This refers especially to the use of compounds of formula (II-1a), in the synthesis of anthranilamide pesticides of formula (A) as defined above, in which R¹ is Cl and R² is CH₃.

In one embodiment, the present invention relates to the use of a compound of formula (II), in the synthesis of anthranilamide pesticides of formula (A), in which
R¹ is Cl, Br, I, or CN;
R² is CH₃, Cl, Br;
R³ is Cl, Br, I, CN, CF₃, CHF₂, OCH₂F, or a residue of formula T:

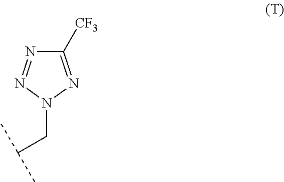

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

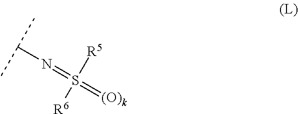

wherein
R⁵, R⁶ are selected independently of one another from the group consisting of methyl, ethyl, isopropyl, n-propyl, n-butyl, isobutyl, tert-butyl, cyclopropyl, cyclopropylmethyl, preferably methyl, ethyl, isopropyl, most preferably ethyl;
k is 0 or 1.

This refers especially to the use of compounds of formula (II-1a), in the synthesis of anthranilamide pesticides of formula (A) as defined above, in which R¹ is Cl and R² is CH₃.

In one embodiment, the present invention relates to the use of a compound of formula (II), in the synthesis of anthranilamide pesticides of formula (A), in which
R^{py} is H;
R^{4a} and R^{4b} together form a group (L)

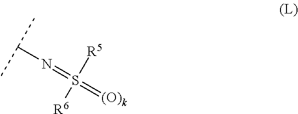

wherein R¹, R², R³, R⁵, R⁶, k are as defined in a compound of Table A′.

This refers especially to the use of compounds of formula (II-1a), in the synthesis of anthranilamide pesticides of formula (A) as defined above, in which R¹ is Cl and R² is CH₃.

TABLE A′

| | R¹ | R² | R³ | R⁵ | R⁶ | k |
|---|---|---|---|---|---|---|
| A′-1 | Cl | CH₃ | CF₃ | C₂H₅ | C₂H₅ | 0 |
| A′-2 | Cl | Cl | CF₃ | C₂H₅ | C₂H₅ | 0 |
| A′-3 | Cl | CH₃ | CF₃ | CH(CH₃)₂ | CH(CH₃)₂ | 0 |
| A′-4 | Cl | Cl | CF₃ | CH(CH₃)₂ | CH(CH₃)₂ | 0 |
| A′-5 | Br | Br | CF₃ | C₂H₅ | C₂H₅ | 0 |

In one embodiment, the present invention relates to the use of a compound of formula (II), in the synthesis of anthranilamide pesticides of formula (A), in which
R^{py} is H;
R^{4a} and R^{4b} together form a group (L)

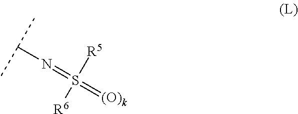

wherein R¹, R², R³, R⁵, R⁶, k are as defined in a compound of Table A″.

This refers especially to the use of compounds of formula (II-1a), in the synthesis of anthranilamide pesticides of formula (A) as defined above, in which R¹ is Cl and R² is CH₃.

TABLE A″

| | R¹ | R² | R³ | R⁵ | R⁶ | k |
|---|---|---|---|---|---|---|
| A″-1 | Cl | CH₃ | CF₃ | C₂H₅ | C₂H₅ | 1 |
| A″-2 | Cl | Cl | CF₃ | C₂H₅ | C₂H₅ | 1 |
| A″-3 | Cl | CH₃ | CF₃ | CH(CH₃)₂ | CH(CH₃)₂ | 1 |
| A″-4 | Cl | Cl | CF₃ | CH(CH₃)₂ | CH(CH₃)₂ | 1 |
| A″-5 | Br | Br | CF₃ | C₂H₅ | C₂H₅ | 1 |

In one embodiment, the present invention relates to the use of a compound of formula (II), in the synthesis of anthranilamide pesticides of formula (A), which are compounds of formula (A-1),

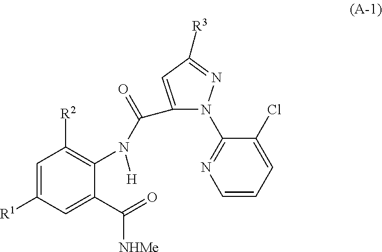

in which
R¹ is Cl, Br, or CN;
R² is CH₃, Cl, Br;
R³ is Br, CF₃, or a residue of formula T:

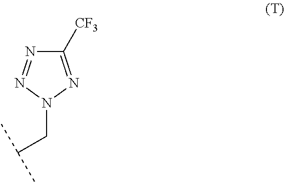

In particular, the present invention relates to the use of a compound of formula (II), in the synthesis of anthranilamide pesticides of formula (A-1), in which
R¹ is Cl, R² is CH₃, and R³ is Br; or
R¹ is CN, R² is CH₃, and R³ is Br; or
R¹ is Cl, R² is CH₃, and R³ is a residue of formula T:

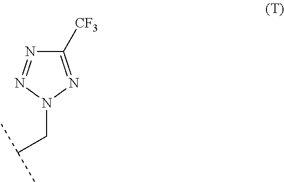

In particular, the present invention relates to the use of a compound of formula (II), in the synthesis of anthranilamide pesticides of formula (A), which are compounds of formula (A-2),

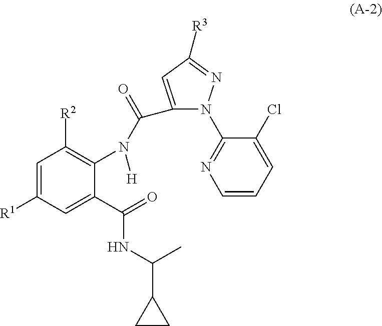

in which
R¹ is Cl, Br, or CN;
R² is CH₃, Cl, or Br;
R³ is Br, CF₃.

In particular, the present invention relates to the use of a compound of formula (II), in the synthesis of anthranilamide pesticides of formula (A-2), in which
R¹ is Cl, R² is Br, and R³ is Br.

In particular, the present invention relates to the use of a compound of formula (II), in the synthesis of anthranilamide pesticides of formula (A), which are compounds of formula (A-4),

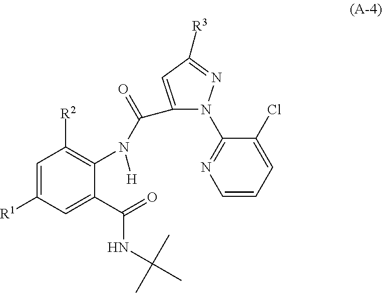

in which
R¹ is Cl, Br, or CN;
R² is CH₃, Cl, or Br;
R³ is Br, OCH₂F or CF₃.

In particular, the present invention relates to the use of a compound of formula (II), in the synthesis of anthranilamide pesticides of formula (A-4), in which
R¹ is Cl, R² is Br, and R³ is OCH₂F.

In particular, the present invention relates to the use of a compound of formula (II), in the synthesis of anthranilamide pesticides of formula (A), which are compounds of formula (A-5),

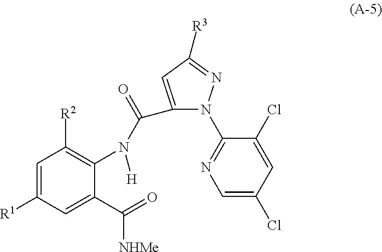

in which
R¹ is Cl, Br, or CN;
R² is CH₃, Cl, or Br;
R³ is Br, or CF₃.

In particular, the present invention relates to the use of a compound of formula (II), in the synthesis of anthranilamide pesticides of formula (A-5), in which
R¹ is Cl, R² is Cl, and R³ is Br.

In particular, the present invention relates to the use of a compound of formula (II), in the synthesis of anthranilamide pesticides of formula (A), which are compounds of formula (A-6),

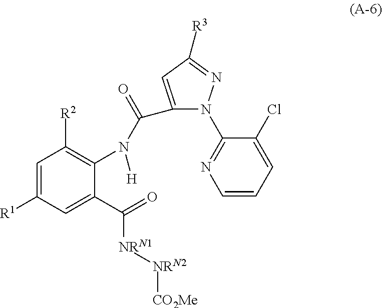

in which
R¹ is Cl, Br, or CN;
R² is CH₃, Cl, or Br;
R³ is Br, or CF₃;
R^{N1} is methyl or ethyl;
R^{N2} is methyl or ethyl.

In particular, the present invention relates to the use of a compound of formula (II), in the synthesis of anthranilamide pesticides of formula (A-6), in which
R¹ is Br, R² is Br, and R³ is Br, and R^{N1} and R^{N2} are as follows:
R^{N1} is hydrogen and R^{N2} is hydrogen; or
R^{N1} is hydrogen and R^{N2} is methyl; or
R^{N1} is methyl and R^{N2} is hydrogen; or
R^{N1} is methyl and R^{N2} is methyl; or
R^{N1} is ethyl and R^{N2} is hydrogen; or
R^{N1} is hydrogen and R^{N2} is ethyl; or
R^{N1} is methyl and R^{N2} is ethyl; or
R^{N1} is ethyl and R^{N2} is methyl; or
R^{N1} is ethyl and R^{N2} is ethyl.

Accordingly, the present invention relates to a process for subsequent reaction of the compounds of formula (I). The invention relates to a process for preparing an anthranilamide compound of formula (A):

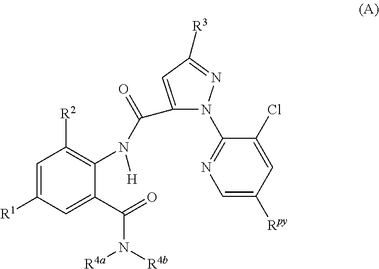

in which
R¹ is Cl, Br, I, or CN;
R² is CH₃, Cl, Br;
R³ is Cl, Br, I, CN, CF₃, CHF₂, OCH₂F or a residue of formula T:

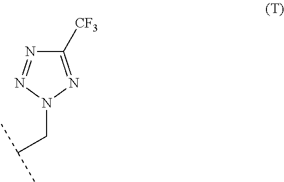

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

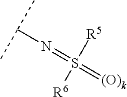

wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
and wherein R^{a}, R^{b}, R^{c}, R^{d}, R^{e}, R^{f} and n are as defined above;
or a stereoisomer, salt, tautomer or N-oxide, or a polymorphic crystalline form, a co-crystal or a solvate of a compound or a stereoisomer, salt, tautomer or N-oxide thereof;
the process comprising
i) providing a compound of the formula (I) by a process as defined herein; or
ii) providing a compound of the formula (II) by a process as defined herein, wherein the compound of formula (II) may be used in the processes leading to compounds of formula (I); or
iii) performing reaction steps starting from or leading via an intermediate compound (II) as defined herein, especially an intermediate compound of formula (II-1a), but also intermediate compounds of formula (II-1b) or (II-1c).

Furthermore, the invention relates to a process for preparing an anthranilamide compound of formula (A-0):

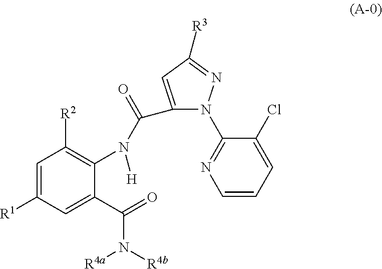

in which
R¹ is Cl, Br, I, or CN;
R² is CH₃, Cl, Br;
R³ is Cl, Br, I, CN, CF₃ or CHF₂;
R^{4a} and R^{4b} are independently selected from hydrogen, C₁-C₄-alkyl,
or
R^{4a} and R^{4b} together form a group (L)

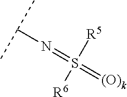

wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
   and
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
   C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
   two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl);
and especially wherein R^{a}, R^{b}, R^{c}, R^{d}, R^{f}, k and n are as defined above;
or a stereoisomer, salt, tautomer or N-oxide, or a polymorphic crystalline form, a co-crystal or a solvate of a compound or a stereoisomer, salt, tautomer or N-oxide thereof;
the process comprising
i) providing a compound of the formula (I) by a process as defined herein; or
ii) providing a compound of the formula (II) by a process as defined herein, wherein the compound of formula (II) may be used in the processes leading to compounds of formula (I); or
iii) performing reaction steps starting from or leading via an intermediate compound (II) as defined herein, especially an intermediate compound of formula (II-1a), but also intermediate compounds of formula (II-1b) or (II-1c).

In one embodiment, the invention relates to saidprocess comprising step i, ii or iii for preparing a compound of formula (A-0) as defined herein, wherein
R¹ is Cl, CN;
R² is CH₃;
R³ is Br, CF₃; and
R^{4a} and R^{4b} are one hydrogen and the other methyl, or
R^{4a} and R^{4b} are one hydrogen and the other cyclopropylmethyl, or
R^{4a} and R^{4b} together form a group (L)

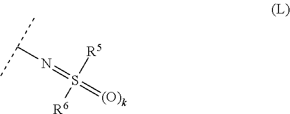

in which R⁵ and R⁶ are identical and selected from methyl, ethyl, isopropyl; and
k is 0.

In particular, the invention relates to said process comprising step i, ii or iii for preparing a compound of formula (A) as defined herein, wherein
R¹ is Cl, Br, CN;
R² is CH₃; Cl, Br;
R³ is Br, CF₃; OCH₂F or a residue of formula T:

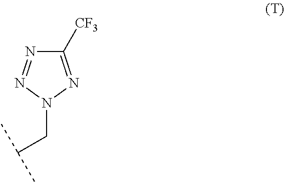

R^{py} is H or Cl;
R^{4a} and R^{4b} are one hydrogen and the other methyl, or
R^{4a} and R^{4b} are one hydrogen and the other cyclopropylmethyl, or
R^{4a} and R^{4b} are one hydrogen and the other cyclopropylethyl, or
R^{4a} and R^{4b} are one hydrogen and the other tert-butyl, or
R^{4a} and R^{4b} are one hydrogen and the other NR^{N2}—CO₂—CH₃, wherein R^{N2} is hydrogen, methyl or ethyl, or
R^{4a} and R^{4b} are one methyl and the other NR^{N2}—CO₂—CH₃, wherein R^{N2} is hydrogen, methyl or ethyl, or
R^{4a} and R^{4b} are one ethyl and the other NR^{N2}—CO₂—CH₃, wherein R^{N2} is hydrogen, methyl or ethyl, or
R^{4a} and R^{4b} together form a group (L)

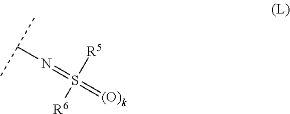

in which R⁵ and R⁶ are identical and selected from methyl, ethyl, isopropyl; and
k is 0.

In one embodiment, the invention relates to said process comprising step i, ii or iii for preparing a compound of formula (A) as defined herein, wherein
R¹ and R² are both Cl or are both Br;
R³ is Br, CF₃;
R^{py} is H or Cl; and
R^{4a} and R^{4b} are one hydrogen and the other methyl, or
R^{4a} and R^{4b} are one hydrogen and the other cyclopropylmethyl, or
R^{4a} and R^{4b} are one hydrogen and the other cyclopropylethyl, or
R^{4a} and R^{4b} are one hydrogen and the other tert-butyl, or
R^{4a} and R^{4b} are one hydrogen and the other NR^{N2}—CO₂—CH₃, wherein R^{N2} is hydrogen, methyl or ethyl, or
R^{4a} and R^{4b} are one methyl and the other NR^{N2}—CO₂—CH₃, wherein R^{N2} is hydrogen, methyl or ethyl, or
R^{4a} and R^{4b} are one ethyl and the other NR^{N2}—CO₂—CH₃, wherein R^{N2} is hydrogen, methyl or ethyl, or
R^{4a} and R^{4b} together form a group (L)

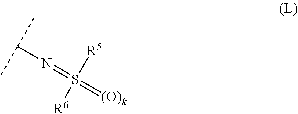

in which R⁵ and R⁶ are identical and selected from methyl, ethyl, isopropyl; and
k is 0.

In one embodiment, the invention relates to said process comprising step i, ii or iii for preparing a compound of formula (A) as defined herein, wherein
R¹ is Cl, Br, I, or CN;
R² is CH₃, Cl, Br;
R³ is Cl, Br, I, CN, CF₃, CHF₂, OCH₂F, or a residue of formula T:

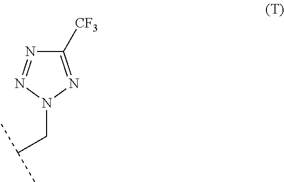

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

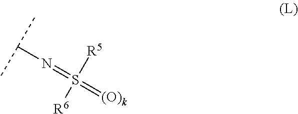

wherein
R⁵, R⁶ are selected independently of one another from the group consisting of hydrogen, C₁-C₄-alkyl, C₃-C₈-cycloalkyl, or
   R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring,
k is 0 or 1.

This refers especially to the processes starting from or leading via compounds of formula (II-1a), for preparing anthranilamide pesticides of formula (A) as defined above, in which R¹ is Cl and R² is CH₃.

In one embodiment, the invention relates to said process comprising step i, ii or iii for preparing a compound of formula (A) as defined herein, wherein
R¹ is Cl, Br, I, or CN;
R² is CH₃, Cl, Br;
R³ is Cl, Br, I, CN, CF₃, CHF₂, OCH₂F, or a residue of formula T:

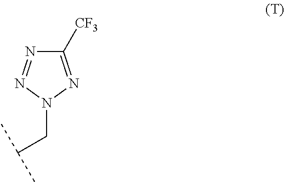

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

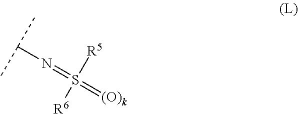

wherein
R⁵, R⁶ are selected independently of one another from the group consisting of hydrogen, C₁-C₄-alkyl and C₃-C₈-cycloalkyl. k is 0 or 1.

This refers especially to the processes starting from or leading via compounds of formula (II-1a), for preparing anthranilamide pesticides of formula (A) as defined above, in which R¹ is Cl and R² is CH₃.

In one embodiment, the invention relates to said process comprising step i, ii or iii for preparing a compound of formula (A) as defined herein, wherein
R¹ is Cl, Br, I, or CN;
R² is CH₃, Cl, Br;
R³ is Cl, Br, I, CN, CF₃, CHF₂, OCH₂F, or a residue of formula T:

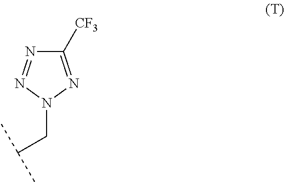

R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl,
or
R^{4a} and R^{4b} together form a group (L)

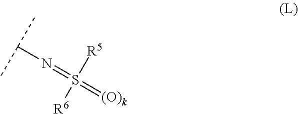

wherein
R⁵, R⁶ are selected independently of one another from the group consisting of methyl, ethyl, isopropyl, n-propyl, n-butyl, isobutyl, tert-butyl, cyclopropyl, cyclopropylmethyl, preferably methyl, ethyl, isopropyl, most preferably ethyl;
k is 0 or 1.

This refers especially to the processes starting from or leading via compounds of formula (II-1a), for preparing anthranilamide pesticides of formula (A) as defined above, in which R¹ is Cl and R² is CH₃.

In one embodiment, the invention relates to said process comprising step i, ii or iii for preparing a compound of formula (A) as defined herein, wherein
R^{py} is H;
R^{4a} and R^{4b} together form a group (L)

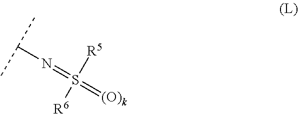

wherein R¹, R², R³, R⁵, R⁶, k are as defined in a compound of Table A′.

This refers especially to the processes starting from or leading via compounds of formula (II-1a), for preparing anthranilamide pesticides of formula (A) as defined above, in which R¹ is Cl and R² is CH₃.

TABLE A′

| | R¹ | R² | R³ | R⁵ | R⁶ | k |
|---|---|---|---|---|---|---|
| A′-1 | Cl | CH₃ | CF₃ | C₂H₅ | C₂H₅ | 0 |
| A′-2 | Cl | Cl | CF₃ | C₂H₅ | C₂H₅ | 0 |
| A′-3 | Cl | CH₃ | CF₃ | CH(CH₃)₂ | CH(CH₃)₂ | 0 |
| A′-4 | Cl | Cl | CF₃ | CH(CH₃)₂ | CH(CH₃)₂ | 0 |
| A′-5 | Br | Br | CF₃ | C₂H₅ | C₂H₅ | 0 |

In one embodiment, the invention relates to said process comprising step i, ii or iii for preparing a compound of formula (A) as defined herein, wherein
R^{py} is H;
R^{4a} and R^{4b} together form a group (L)

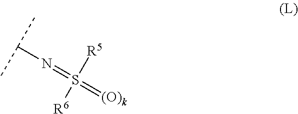

wherein R¹, R², R³, R⁵, R⁶, k are as defined in a compound of Table A″.

This refers especially to the processes starting from or leading via compounds of formula (II-1a), for preparing anthranilamide pesticides of formula (A) as defined above, in which R¹ is Cl and R² is CH₃.

TABLE A″

| | R¹ | R² | R³ | R⁵ | R⁶ | k |
|---|---|---|---|---|---|---|
| A″-1 | Cl | CH₃ | CF₃ | C₂H₅ | C₂H₅ | 1 |
| A″-2 | Cl | Cl | CF₃ | C₂H₅ | C₂H₅ | 1 |
| A″-3 | Cl | CH₃ | CF₃ | CH(CH₃)₂ | CH(CH₃)₂ | 1 |
| A″-4 | Cl | Cl | CF₃ | CH(CH₃)₂ | CH(CH₃)₂ | 1 |
| A″-5 | Br | Br | CF₃ | C₂H₅ | C₂H₅ | 1 |

In one embodiment, the invention relates to said process comprising step i, ii or iii for preparing a compound of formula (A) as defined herein, which are compounds of formula (A-1) as defined above, in which
R¹ is Cl, Br, or CN;
R² is CH₃, Cl, Br;
R³ is Br, CF₃, or a residue of formula T:

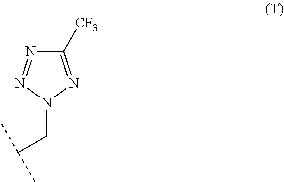

In particular, the present invention relates to said processes comprising step i, ii or iii for preparing anthranilamide pesticides of formula (A-1) as defined above, in which
R¹ is Cl, R² is CH₃, and R³ is Br; or
R¹ is CN, R² is CH₃, and R³ is Br; or
R¹ is Cl, R² is CH₃, and R³ is a residue of formula T:

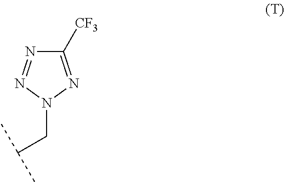

In one embodiment, the invention relates to said process comprising step i, ii or iii for preparing a compound of formula (A) as defined herein, which are compounds of formula (A-2) as defined above, in which
R¹ is Cl, Br, or CN;
R² is CH₃, Cl, or Br;
R³ is Br, CF₃.

In particular, the present invention relates to said processes comprising step i, ii or iii for preparing anthranilamide pesticides of formula (A-2) as defined above, in which
R¹ is Cl, R² is Br, and R³ is Br.

In one embodiment, the invention relates to said process comprising step i, ii or iii for preparing a compound of formula (A) as defined herein, which are compounds of formula (A-4) as defined above, in which
R¹ is Cl, Br, or CN;
R² is CH₃, Cl, or Br;
R³ is Br, OCH₂F or CF₃.

In particular, the present invention relates to said processes comprising step i, ii or iii for preparing anthranilamide pesticides of formula (A-4) as defined above, in which
R¹ is Cl, R² is Br, and R³ is OCH₂F.

In one embodiment, the invention relates to said process comprising step i, ii or iii for preparing a compound of formula (A) as defined herein, which are compounds of formula (A-5) as defined above, in which
R¹ is Cl, Br, or CN;
R² is CH₃, Cl, or Br;
R³ is Br, or CF₃.

In particular, the present invention relates to said processes comprising step i, ii or iii for preparing anthranilamide pesticides of formula (A-5) as defined above, in which
R¹ is Cl, R² is Cl, and R³ is Br.

In one embodiment, the invention relates to said process comprising step i, ii or iii for preparing a compound of formula (A) as defined herein, which are compounds of formula (A-6) as defined above, in which
R¹ is Cl, Br, or CN;
R² is CH₃, Cl, or Br;
R³ is Br, or CF₃;
R^{N1} is methyl or ethyl;
R^{N2} is methyl or ethyl.

In particular, the present invention relates to said processes comprising step i, ii or iii for preparing anthranilamide pesticides of formula (A-6), in which
R¹ is Br, R² is Br, and R³ is Br, and R^{N1} and R^{N2} are as follows:
R^{N1} is hydrogen and R^{N2} is hydrogen; or
R^{N1} is hydrogen and R^{N2} is methyl; or
R^{N1} is methyl and R^{N2} is hydrogen; or
R^{N1} is methyl and R^{N2} is methyl; or
R^{N1} is ethyl and R^{N2} is hydrogen; or
R^{N1} is hydrogen and R^{N2} is ethyl; or
R^{N1} is methyl and R^{N2} is ethyl; or
R^{N1} is ethyl and R^{N2} is methyl; or
R^{N1} is ethyl and R^{N2} is ethyl.

In the following, possible routes to compounds of formula (A) are described, starting from compounds of formula (I). Nevertheless, the use of compounds of formula (I) is not limited to these processes. Compounds of formula (A) may be obtained also via alternative ways, but nevertheless using compounds of formula (I) as described herein.

In one embodiment, wherein R^{4a} and R^{4b} together form a group (L)

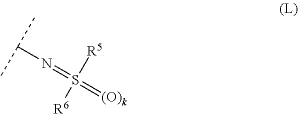

in which R⁵ and R⁶ are identical and selected from methyl, ethyl, isopropyl; and

k is 0,

the compounds of formula (I) are converted to compounds of formula (V)

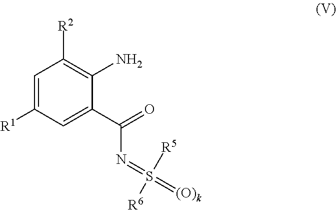

in which the variables R¹, R², R⁵, R⁶ and k are each as defined herein.

This conversion is described e.g. in WO2013/024008 or WO2007/006670.

Compounds of formula (V) may be coupled with pyridylpyrazole acids of formula (VI)

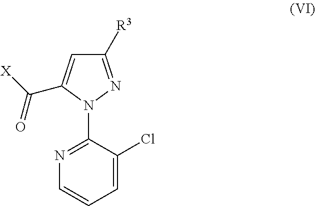

wherein
R³ is as defined herein;
X is selected from halogen, preferably Cl, OH, O—Mg—Cl, O—Mg—Br, imidazole, —O—CO—R^{x}, —O—CO—OR^{x}, —OSO₂R^{x}, —SR^{y}, in which
R^{x} is independently selected from C₁-C₆-alkyl, trifluoromethyl and phenyl which is optionally substituted with C₁-C₆-alkyl (preferably as o-toluene, m-toluene, p-toluene, o-xylene, m-xylene, p-xylene) or halogen, and
R^{y} is independently selected from C₁-C₆-alkyl and phenyl which is optionally substituted with C₁-C₆-alkyl (preferably as o-toluene, m-toluene, p-toluene, o-xylene, m-xylene, p-xylene) or halogen.

thus forming anthranilamide pesticide compounds of formula (A) as described above.

For preparation of substituted 1-pyridin-2-yl-1H-pyrazole-5-carbonylchlorides of formula (VI), a process described in WO 02/070483, WO03/015519, WO 07/043677 and WO 08/130021 has been found to be useful. Especially useful preparation methods are described in WO2013/024007 and in WO2013/076092, and also in PCT/EP2014/062709 and in PCT/EP2014/060082. The reactions of all these publications are understood to be part of this invention, if they are combined with the reaction steps according to the present invention.

As said above, the invention relates to combinations of process steps, comprising step (a) and/or step (b), especially processes which lead to anthranilamide compounds of formula (A) as defined herein.

Accordingly, in a further aspect, the present invention relates to a process for preparing an anthranilamide compound of formula (A) as described herein, especially a compound of formula (A), wherein
R¹ is Cl, CN;
R² is CH₃;
R³ is Br, CF₃; or a residue of formula T:

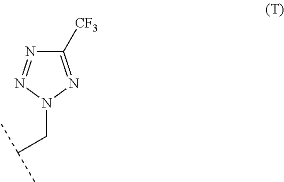

and
R^{4a} and R^{4b} are one hydrogen and the other methyl, or
R^{4a} and R^{4b} are one hydrogen and the other cyclopropylmethyl, or
R^{4a} and R^{4b} together form a group (L)

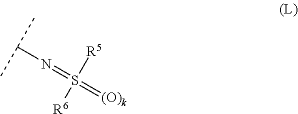

in which R⁵ and R⁶ are identical and selected from methyl, ethyl, isopropyl; and
k is 0.

and wherein the process comprises
i) providing a compound of the formula (I) by a process as described herein, and
ii) converting the compound of formula (I) to a compound of formula (A), optionally via conversion to a compound of formula (V) as described herein, and optionally via coupling with the corresponding carbonyl compound of formula (VI) as described herein.

In a further aspect, the present invention relates to a process for preparing an anthranilamide precursor compound of formula (V), wherein the process comprises
i) providing a compound of the formula (I) by a process as described herein,
ii-1) reacting the compound of formula (I) to a compound of formula (V) as described herein.
wherein the variables are as defined herein.

In a further aspect, the present invention relates to a process for preparing an anthranilamide compound of formula (A), wherein the process comprises
ii) providing a compound of the formula (I) by a process as described herein,
ii-1) reacting the compound of formula (I) to a compound of formula (V) as described herein, and
ii-2) coupling the compound of formula (V) with a carbonyl compound of formula (VI) as described herein.

In the context of the present invention, the terms used generically are each defined as follows: The prefix Cₓ-C_{y} refers in the particular case to the number of possible carbon atoms. The term “halogen” denotes in each case fluorine, bromine, chlorine or iodine, in particular fluorine, chlorine or bromine.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “alkyl” as used herein (and in the alkyl moieties of other groups comprising an alkyl group, e.g. alkoxy, alkylcarbonyl, alkylthio, alkylsulfinyl, alkylsulfonyl and alkoxyalkyl) denotes in each case a straight-chain or branched alkyl group having usually from 1 to 10 carbon atoms, frequently from 1 to 6 carbon atoms, preferably 1 to 4 carbon atoms and in particular from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl and 2-propylpentyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy and haloalkylthio) denotes in each case a straight-chain or branched alkyl group having usually from 1 to 10 carbon atoms, frequently from 1 to 6 carbon atoms, wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “fluoroalkyl”, as used herein (and in the fluoroalkyl units of fluoroalkoxy, fluoroalkylthio, fluoroalkylsulfinyl and fluoroalkylsulfonyl) denotes in each case straight-chain or branched alkyl groups having usually from 1 to 10 carbon atoms, frequently from 1 to 6 carbon atoms and in particular 1 to 4 carbon atoms, wherein the hydrogen atoms of this group are partially or totally replaced with fluorine atoms. Examples thereof are fluoromethyl, difluoromethyl, trifluoromethyl, 1-fluoroethyl, 2-fluoroethyl, 2,2-difluoroethyl, 2,2,2-trifluoroethyl, pentafluoroethyl, 3,3,3-trifluoroprop-1-yl, 1,1,1-trifluoroprop-2-yl, heptafluoroisopropyl, 1-fluorobutyl, 2-fluorobutyl, 3-fluorobutyl, 4-fluorobutyl, 4,4,4-trifluorobutyl, fluoro-tert-butyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms, 3 to 8 carbon atoms or 3 to 6 carbon atoms, such as cyclopropyl, cyclobutyl, cyclopentyl, cyclohexyl, cycloheptyl, cyclooctyl, bicyclo[2.1.1]hexyl, bicyclo[3.1.1]heptyl, bicyclo[2.2.1]heptyl, and bicyclo[2.2.2]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, 3 to 8 carbon atoms or 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “fluorocylcoalkyl” as used herein, denotes a halocycloalkyl radical, as defined above, wherein the one or more halogen atoms are fluorine atoms.

The term “alkenyl” as used herein denotes in each case a singly unsaturated hydrocarbon radical having usually 2 to 10, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “alkenylene” (or alkenediyl) as used herein in each case denotes an alkenyl radical as defined above, wherein one hydrogen atom at any position of the carbon backbone is replaced by one further binding site, thus forming a bivalent moiety.

The term “haloalkenyl” as used herein, which may also be expressed as “alkenyl which may be substituted by halogen”, and the haloalkenyl moieties in haloalkenyloxy, haloalkenylcarbonyl and the like refers to unsaturated straight-chain or branched hydrocarbon radicals having 2 to 10 (“C₂-C₁₀-haloalkenyl”) or 2 to 6 (“C₂-C₆-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “fluoroalkenyl” as used herein, denotes a haloalkenyl radical, as defined above, wherein the one or more halogen atoms are fluorine atoms.

The term “alkynyl” as used herein denotes unsaturated straight-chain or branched hydrocarbon radicals having usually 2 to 10, frequently 2 to 6, preferably 2 to 4 carbon atoms and one or two triple bonds in any position, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “alkynylene” (or alkynediyl) as used herein in each case denotes an alkynyl radical as defined above, wherein one hydrogen atom at any position of the carbon backbone is replaced by one further binding site, thus forming a bivalent moiety.

The term “haloalkynyl” as used herein, which is also expressed as “alkynyl which may be substituted by halogen”, refers to unsaturated straight-chain or branched hydrocarbon radicals having usually 3 to 10 carbon atoms, frequently 2 to 6, preferably 2 to 4 carbon atoms, and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 10 carbon atoms, frequently from 1 to 6 carbon atoms, preferably 1 to 4 carbon atoms, which is bound to the remainder of the molecule via an oxygen atom. Examples of an alkoxy group are methoxy, ethoxy, n-propoxy, iso-propoxy, n-butyloxy, 2-butyloxy, iso-butyloxy, tert-butyloxy, and the like.

The term “haloalkoxy” as used herein denotes in each case a straight-chain or branched alkoxy group, as defined above, having from 1 to 10 carbon atoms, frequently from 1 to 6 carbon atoms, preferably 1 to 4 carbon atoms, preferably 1 to 3 carbon atoms, wherein the hydrogen atoms of this group are partially or totally replaced with halogen atoms, in particular fluorine atoms. Preferred haloalkoxy moieties include C₁-C₄-haloalkoxy, in particular halomethoxy, and also in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2-dichloro-2-fluorethoxy, 2,2,2-trichloroethoxy, pentafluoroethoxy and the like.

The term “alkoxy-alkyl” as used herein denotes in each case alkyl usually comprising 1 to 6 carbon atoms, preferably 1 to 4 carbon atoms, wherein 1 carbon atom carries an alkoxy radical usually comprising 1 to 10, frequently 1 to 6, in particular 1 to 4, carbon atoms as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “fluoroalkoxy-alkyl” as used herein denotes in each case alkyl as defined above, usually comprising 1 to 6 carbon atoms, preferably 1 to 4 carbon atoms, wherein 1 carbon atom carries an fluoroalkoxy radical as defined above, usually comprising 1 to 10, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or alkyl-S—)” as used herein denotes in each case a straight-chain or branched saturated alkyl group as defined above, usually comprising 1 to 10 carbon atoms, frequently comprising 1 to 6 carbon atoms, preferably 1 to 4 carbon atoms, which is attached via a sulfur atom at any position in the alkyl group. Examples are methylthio, ethylthio, n-propylthio, iso-propylthio, n-butylthio, 2-butylthio, iso-butylthio, tert-butylthio, and the like.

The term “haloalkylthio” as used herein refers to an alkylthio group as defined above wherein the hydrogen atoms are partially or fully substituted by fluorine, chlorine, bromine and/or iodine. Examples are fluoromethylthio, difluoromethylthio, trifluoromethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoro-ethylthio, 2,2-dichloro-2-fluorethylthio, 2,2,2-trichloroethylthio, pentafluoroethylthio and the like

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₆-alkylsulfinyl” refers to a C₁-C₆-alkyl group, as defined above, attached via a sulfinyl [S(O)] group. Examples are methylsulfinyl, ethylsulfinyl, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl), 1,1-dimethylethylsulfinyl (tert-butylsulfinyl), pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. For example, the term “C₁-C₆-alkylsulfonyl” refers to a C₁-C₆-alkyl group, as defined above, attached via a sulfonyl [S(O)₂] group. Examples are methylsulfonyl, ethylsulfonyl, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl), 1,1-dimethylethylsulfonyl (tert-butylsulfonyl), pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

The term “alkylamino” as used herein denotes in each case a group —NHR, wherein R is a straight-chain or branched alkyl group usually having from 1 to 6 carbon atoms, preferably 1 to 4 carbon atoms. Examples of an alkylamino group are methylamino, ethylamino, n-propylamino, isopropylamino, n-butylamino, 2-butylamino, iso-butylamino, tert-butylamino, and the like.

The term “dialkylannino” as used herein denotes in each case a group —NRR′, wherein R and R′, independently of each other, are a straight-chain or branched alkyl group each usually having from 1 to 6 carbon atoms, preferably 1 to 4 carbon atoms. Examples of a dialkylamino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, alkoxycarbonyl and haloalkoxycarbonyl.

The term “aryl” as used herein refers to a mono-, bi- or tricyclic aromatic hydrocarbon radical having 6 to 14 carbon atoms. Examples thereof comprise phenyl, naphthyl, fluorenyl, azulenyl, anthracenyl and phenanthrenyl. Aryl is preferably phenyl or naphthyl and especially phenyl.

The term “3-, 4-, 5-, 6-, 7- or 8-membered saturated carbocyclic ring” as used herein refers to carbocyclic rings, which are monocyclic and fully saturated. Examples of such rings include cyclopropane, cyclobutane, cyclopentane, cyclohexane, cycloheptane, cyclooctane and the like.

The terms “3-, 4-, 5-, 6-, 7- or 8-membered partially unsaturated carbocyclic ring” and “5- or 6-membered partially unsaturated carbocyclic ring” refer to carbocyclic rings, which are monocyclic and have one or more degrees of unsaturation. Examples of such rings include cyclopropene, cyclobutene, cyclopentene, cyclohexene, cycloheptene, cyclooctene and the like.

The term “3-, 4-, 5-, 6- or 7-membered saturated, partially unsaturated or completely unsaturated heterocyclic ring containing 1, 2 or 3 heteroatoms or heteroatom groups selected from N, O, S, NO, SO and SO₂, as ring members” [wherein “completely/fully unsaturated” includes also “aromatic”] as used herein denotes monocyclic radicals, the monocyclic radicals being saturated, partially unsaturated or fully unsaturated (including aromatic). The heterocyclic ring may be attached to the remainder of the molecule via a carbon ring member or via a nitrogen ring member.

Examples of a 3-, 4-, 5-, 6- or 7-membered saturated heterocyclic ring include: oxiranyl, aziridinyl, azetidinyl, tetrahydrofuran-2-yl, tetrahydrofuran-3-yl, tetrahydrothien-2-yl, tetrahydrothien-3-yl, pyrrolidin-2-yl, pyrrolidin-3-yl, pyrazolidin-3-yl, pyrazolidin-4-yl, pyrazolidin-5-yl, imidazolidin-2-yl, imidazolidin-4-yl, oxazolidin-2-yl, oxazolidin-4-yl, oxazolidin-5-yl, isoxazolidin-3-yl, isoxazolidin-4-yl, isoxazolidin-5-yl, thiazolidin-2-yl, thiazolidin-4-yl, thiazolidin-5-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl, morpholin-2-yl, morpholin-3-yl, thiomorpholin-2-yl, thionnorpholin-3-yl, 1-oxothiomorpholin-2-yl, 1-oxothiomorpholin-3-yl, 1,1-dioxothionnorpholin-2-yl, 1,1-dioxothionnorpholin-3-yl, azepan-1-, -2-, -3- or -4-yl, oxepan-2-, -3-, 4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl. A 3-, 4-, 5-, 6- or 7-membered completely unsaturated (including aromatic) heterocyclic ring is e.g. a 5- or 6-membered fully unsaturated (including aromatic) heterocyclic ring. Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 4-isoxazolyl, 2-thiazolyl, 4-thiazolyl, 5-thiazolyl, 4-isothiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members” as used herein denotes a saturated or unsaturated 3- to 8-membered ring system which optionally contains 1 to 3 heteroatoms selected from N, O, S, NO, SO and SO₂, as defined above, with the exception of the completely unsaturated ring systems.

Preferences

The remarks made below concerning preferred embodiments of the variables of the compounds of the formulae (I), (II), (III), (IV), (A), and their subvariants are valid on their own as well as preferably in combination with each other as well as concerning the uses and processes according to the invention.

In one embodiment of the invention, R³ is CF₃. Especially, in the compounds of the formula (I), (II), (III), (IV), (A), and their subvariants, and the processes related to them, R³ is CF₃.

In one embodiment of the invention, R³ is CHF₂. Especially, in the compounds of the formula (I), (II), (III), (IV), (A), and their subvariants, and the processes related to them, R³ is CHF₂.

In one embodiment of the invention, R³ is a residue of formula T.

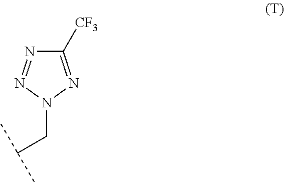

Especially, in the compounds of the formula (I), (II), (III), (IV), (A), and their subvariants, and the processes related to them, R³ is the residue of formula T.

In the compounds of the formulae (I), (II), (III), (A), and their subvariants, R¹ is hydrogen, halogen, halomethyl or cyano, preferably, R¹ is Cl or Br or cyano, most preferably Cl. R² is selected from the group consisting of halogen, methyl and halomethyl; preferably from methyl, Cl, Br; most preferably methyl.

In the compounds of the formulae (A) and (V), k is preferably 0.

In the compounds of the formulae (A) and (V), wherein k is 0, R⁵ and R⁶ are preferably, independently of each other, selected from hydrogen, C₁-C₆-alkyl, C₁-C₆-haloalkyl, C₃-C₆-cycloalkyl, C₃-C₆-halocycloalkyl, C₂-C₄-alkenyl, C₂-C₄-haloalkenyl, wherein the six last radicals may optionally be substituted by one or more radicals R^{a};

or R⁶ and R⁷ together represent a C₄-C₅-alkylene or C₄-C₅-alkenylene chain forming together with the sulfur atom to which they are attached a 5- or 6-membered saturated or partially unsaturated ring, wherein one of the CH₂ groups in the C₄-C₅-alkylene chain or one of the CH₂ or CH groups in the C₄-C₅-alkenylene chain may be replaced by a group independently selected from O, S and N and NH, and wherein the carbon and/or nitrogen atoms in the C₄-C₅-alkylene or C₄-C₅-alkenylene chain may be substituted with 1 or 2 substituents independently selected from halogen, cyano, C₁-C₄-alkyl, C₁-C₄-haloalkyl, C₁-C₄-alkoxy, C₁-C₄-haloalkoxy.

More preferably R⁵ and R⁶ are independently selected from C₁-C₆-alkyl, C₁-C₆-haloalkyl, or R⁵ and R⁶ together represent a C₄-C₅-alkylene chain forming together with the sulfur atom to which they are attached a 5- or 6-membered ring. Particularly preferred R⁵ and R⁶ are each C₁-C₆-alkyl, or together represent a C₄-C₅-alkylene chain forming together with the sulfur atom to which they are attached a 5- or 6-membered ring. More preferably R⁵ and R⁶ are independently selected from C₁-C₄-alkyl, C₁-C₄-haloalkyl, or R⁵ and R⁶ together represent a C₄-C₅-alkylene chain forming together with the sulfur atom to which they are attached a 5- or 6-membered ring. Particularly preferred R⁵ and R⁶ are each C₁-C₄-alkyl, or together represent a C₄-C₅-alkylene chain forming together with the sulfur atom to which they are attached a 5- or 6-membered ring. Particularly preferred, when t is 0, R⁵ and R⁶ are selected independently of one another from C₁-C₆-alkyl, or R⁵ and R⁶ together represent a C₃-C₆-alkylene chain forming together with the sulfur atom to which they are attached a 4-, 5-, 6- or 7-membered saturated ring. Specifically R⁵ and R⁶ are each methyl, isopropyl or ethyl, or together represent a butylene chain forming together with the sulfur atom to which they are attached a 5-membered ring.

In the compounds of the formulae (A) and (V), wherein k is 1, the preferred meanings of R⁵ and R⁶ are the preferred meanings as described above in the compounds of the formulae (VI) and (VII), wherein t is 0.

In this context, the variables R^{a}, R^{b}, R^{c}, R^{d}, R^{b1}, R^{c1}, R^{d1}, R^{e}, R^{f}, R^{g}, R^{h}, Rⁱ, m and n, independently of each other, preferably have one of the following meanings:

R^{a} is selected from C₁-C₄-alkyl, C₁-C₄-fluoroalkyl, C₃-C₆-cycloalkyl, C₃-C₆-fluorocycloalkyl, C₂-C₄-alkenyl, C₂-C₄-fluoroalkenyl, C₁-C₄-alkoxy, Crat-alkylthio, amino, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, where phenyl and the heterocyclic ring may be substituted by 1, 2 or 3 radicals selected from C₁-C₄-alkyl, C₁-C₄-fluoroalkyl, C₅-C₆-cycloalkyl and C₅-C₆-fluorocycloalkyl.

More preferably R^{a} is selected from C₁-C₄-alkyl and C₁-C₄-fluoroalkyl, C₁-C₄-alkoxy, di-(C₁-C₄-alkyl)-amino, phenyl and a 5- or 6-membered saturated, partially unsaturated or completely unsaturated heterocyclic ring containing 1 or 2 heteroatoms selected from N, O and S, as ring members, and in particular selected from C₁-C₃-alkyl and C₁-C₂-fluoroalkyl and C₁-C₂-alkoxy.

R^{b} is selected from C₁-C₄-alkyl, C₁-C₄-fluoroalkyl, C₅-C₆-cycloalkyl, C₅-C₆-fluorocycloalkyl, C₁-C₄-alkoxy-C₁-C₄-alkyl, C₁-C₄-fluoroalkoxy-C₁-C₄-alkyl, phenyl-C₁-C₄-alkyl, phenoxy-C₁-C₄-alkyl and pyridyl-C₁-C₄-alkyl, wherein phenyl and pyridyl in the three last mentioned radicals may optionally carry 1 or 2 radicals selected from halogen, substituents C₁-C₄-alkyl, C₁-C₂-fluoroalkyl, C₁-C₄-alkoxy and C₁-C₂-fluoroalkoxy.

More preferably R^{b} is selected from C₁-C₄-alkyl, C₁-C₄-fluoroalkyl and benzyl, and in particular selected from C₁-C₃-alkyl, C₁-C₂-fluoroalkyl and benzyl.

R^{c}, R^{d} are, independently from one another and independently of each occurrence, selected from C₁-C₄-alkyl, C₁-C₄-fluoroalkyl, C₅-C₆-cycloalkyl, C₅-C₆-fluorocycloalkyl, wherein the four last mentioned radicals may optionally carry 1 or 2 radicals selected from C₁-C₄-alkoxy, C₁-C₄-fluoroalkoxy, C₁-C₄-alkylthio, C₁-C₄-fluoroalkylthio, phenyl, benzyl, pyridyl and phenoxy, wherein the four last mentioned radicals may carry 1 or 2 substituents selected from halogen, C₁-C₄-alkyl, C₁-C₂-fluoroalkyl, C₁-C₄-alkoxy and C₁-C₂-fluoroalkoxy; or R^{c} and R^{d}, together with the nitrogen atom to which they are bound, form a 5- or 6-membered saturated, partly unsaturated or completely unsaturated heterocyclic ring which may contain 1 further heteroatom selected from N, O and S as ring members, where the heterocyclic ring may carry 1 or 2 substituents selected from halogen, C₁-C₄-alkyl and C₁-C₄-fluoroalkyl.

More preferably R^{c}, R^{d} are, independently from one another and independently of each occurrence, selected from C₁-C₄-alkyl, C₁-C₄-fluoroalkyl and benzyl, or R^{c} and R^{d}, together with the nitrogen atom to which they are bound, form a 5- or 6-membered saturated or partly unsaturated heterocyclic ring. In particular, R^{c}, R^{d} are, independently from one another and independently of each occurrence, C₁-C₃-alkyl, C₁-C₂-fluoroalkyl, benzyl, or together with the nitrogen atom to which they are bound form a pyrrolidine or a piperidine ring.

R^{b1} is hydrogen or has one of the preferred meanings given for R^{c}.

R^{c1} is hydrogen or has one of the preferred meanings given for R^{c}.

R^{d1} is hydrogen or has one of the preferred meanings given for R^{d}.

R^{e} is selected from halogen, C₁-C₄-alkyl, C₁-C₄-fluoroalkyl, C₂-C₄-alkenyl, C₂-C₄-fluoroalkenyl, where the four last mentioned radicals may optionally carry 1 or 2 radicals selected from C₁-C₂-alkoxy; C₁-C₄-alkoxy, C₁-C₄-fluoroalkoxy, phenyl, benzyl, pyridyl and phenoxy, wherein the four last mentioned radicals may carry 1 or 2 substituents selected from halogen, C₁-C₂-alkyl and C₁-C₂-fluoroalkyl.

More preferably R^{e} is selected from C₁-C₄-alkyl, C₁-C₄-fluoroalkyl, C₁-C₄-alkoxy and C₁-C₄-fluoroalkoxy, and in particular from C₁-C₃-alkyl, C₁-C₂-fluoroalkyl, C₁-C₂-alkoxy, C₁-C₂-fluoroalkoxy.

R^{f}, R^{g} are, independently of each other and independently of each occurrence, selected from C₁-C₄-alkyl, C₅-C₆-cycloalkyl, C₁-C₂-alkoxy-C₁-C₂-alkyl, phenyl and benzyl.

More preferably R^{f}, R^{g} are, independently of each other and independently of each occurrence, selected from C₁-C₄-alkyl, C₅-C₆-cycloalkyl, benzyl and phenyl, and in particular from C₁-C₃-alkyl, benzyl and phenyl.

R^{h}, Rⁱ are, independently from one another and independently of each occurrence, selected from hydrogen, halogen, C₁-C₄-alkyl, C₁-C₄-fluoroalkyl, C₅-C₆-cycloalkyl, C₅-C₆-fluorocycloalkyl, where the four last mentioned radicals may optionally carry 1 or 2 radicals selected from C₁-C₃-alkyl and C₁-C₃-fluoroalkyl; C₁-C₄-alkoxy, C₁-C₄-fluoroalkoxy, phenyl, pyridyl and phenoxy.

More preferably R^{h}, Rⁱ are, independently of each other and independently of each occurrence, selected from hydrogen, C₁-C₃-alkyl and C₁-C₂-fluoroalkyl.

m is 1 or 2, wherein, in the case of several occurrences, m may be identical or different. More preferably m is 2.

n is 1 or 2, wherein, in the case of several occurrences, n may be identical or different. More preferably n is 2.

### EXAMPLES

The compounds can be characterized e.g. by High Performance Liquid Chromatography, by ¹H-/¹³C-NMR and/or by their melting or boiling points. The following analytical procedures were employed:

Analytical HPLC column: Zorbax Eclipse XDB-C18 1.8 μm 50*4.6 mm von Agilent® Elution: acetonitrile/water+0.1 Vol % H₃PO₄ in a ratio of from 25:75 increasing to 100:0 (7 min) and 100:0 (2 min) at 30° C., UV detection at 208 nm.

¹H-/¹³C-NMR. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singulett.

m.p. is melting point, b.p. is boiling point.

Room temperature means usually 20-25° C.

Starting Materials

2-amino-5-chloro-3-methyl benzoic acid was purchased from WuXi AppTec (Tianjin) Co., Ltd. Phenyl chloroformate was purchased from TCI Fine Chemicals.

### Example 1

### 6-chloro-8-methyl-1H-3,1-benzoxazine-2,4-dione

Phenyl chloroformate (99.2 g, calc. 100 wt-%) was dissolved in THF (80 g) at 25° C. A solution of 2-amino-5-chloro-3-methyl benzoic acid (98.7 g, calc. 100 wt-%) in THF (520 g) was added. The reaction mixture was heated to 50° C. After 6 h the reaction mixture was evaporated at 80° C./2 mbar. The solid residue was suspended in 300 g toluene at 80° C. The suspension was cooled down to 5° C. The solid product was filtered, washed with 100 g of cold toluene and dried in vacuum dryer at 50° C./20 mbar overnight. 109 g product with a purity of 93 wt-% (quant HPLC) were obtained, i.e. a yield of 90.2%. 1H-NMR (400 MHz, ACN-D3): δ/ppm=2.3 (s, 3H), 7.58 (s, 1H), 7.86 (s, 1H), 8.82 (s, broad, 1H), 8.65 (d, 1H)

### Characterisation of the Intermediary Formed 5-chloro-3-methyl-2-(phenoxycarbonylamino)benzoic Acid

The compound was isolated as the main component of the reaction mixture of an experiment conducted similar to example 1 before heating to 50° C. by evaporation of a sample under vacuum.

13C-NMR (125 MHz, THF-D8): δ/ppm=18.7 (q), 122.39 (d, 2C), 125.73 (d), 127.98 (s), 128.95 (d), 129.75 (d, 2C), 131.16 (s), 134.99 (d), 136.76 (s), 138.80 (s), 152.44 (s), 152.84 (s), 167.68 (s)

1H-NMR (400 MHz, DMSO): δ/ppm=2.34 (s, 3H), 7.1-7.2 (m, 3H), 7.25-7.35 (m, 2H), 7.46 (s, 1H), 7.80 (s, 1H), 7.92 (s, broad, 1H), 9.18 (s, broad, 1H)

### Example 2

### 6-chloro-8-methyl-1H-3,1-benzoxazine-2,4-dione

2-Amino-5-chloro-3-methyl benzoic acid (40 g, calc. 100 wt-%) was dissolved in toluene (180 g). Phenyl chloroformate (40.5 g, calc. 100 wt-%) was added at 25° C. The reaction mixture was heated to reflux and kept at reflux for 3 hours. Then the reaction mixture was cooled down to 5° C. The solid product was filtered, washed with 100 g of cold toluene and dried in vacuum dryer at 50° C./120 mbar overnight. 43.5 g product with a purity of 97.5 wt-% (quant HPLC) were obtained, i.e. a yield of 93.0%.

1H-NMR (400 MHz, ACN-D3): δ/ppm=2.3 (s, 3H), 7.58 (s, 1H), 7.86 (s, 1H), 8.82 (s, broad, 1H)

### Example 3

### 6-chloro-8-methyl-1H-3,1-benzoxazine-2,4-dione

2-Amino-5-chloro-3-methyl benzoic acid (98 g, calc. 100 wt-%) was suspended in toluene (335 g). Phenyl chloroformate (99.3 g, calc. 100 wt-%) was added at 25° C. The reaction mixture was stirred at 25° C. for 1 h and then heated to reflux and kept for 2.5 h at reflux. During the conversion time HPLC samples were drawn to monitor the conversion via 5-chloro-3-methyl-2-(phenoxycarbonylamino)benzoic acid (intermediate) to the product. The following data were collected:

| Conversion | Reaction | Starting | Inter- | Product | |
|---|---|---|---|---|---|
| time | temperature | material | mediate | [area- | |
| [h] | [° C.] | [area-%] | [area-%] | %] | Remarks |
| 1 | 25 | 22.1 | 3.2 | n.d. | |
| 2 | 108 | 8.2 | 5.2 | 7.4 | during |
| | | | | | heating |
| 2.75 | 112 | 0.75 | 2.2 | 9.9 | Reflux |
| 3.5 | 112 | n.d. | n.d. | 12.0 | Reflux |

After the last sample was assayed the reaction mixture was cooled down to 5° C. The solid product was filtered, washed with 100 g of cold toluene and dried in vacuum dryer at 50° C./20 mbar overnight. 107.7 g product with a purity of 95.6 wt-% (quant HPLC) were obtained, i.e. a yield of 92.2%.

1H-NMR (400 MHz, THF-D8): δ/ppm=2.37 (s, 3H), 7.58 (s, 1H), 7.82 (s, 1H), 10.1 (s, broad, 1H), 8.65 (d, 1H)

13C-NMR (125 MHz, THF-D8): δ/ppm=16.79 (q), 113.01 (s), 126.84 (d), 127.41 (s), 128.47 (s), 137.85 (d), 139.80 (s), 147.47 (s), 159.45 (s)

### Example 4

### 6-chloro-8-methyl-1H-3,1-benzoxazine-2,4-dione

Phenyl chloroformate (32.5 g, calc. 100 wt-%) was dissolved in THF (39 g) at 25° C. A solution of 2-amino-5-chloro-3-methyl benzoic acid (32 g, calc. 100 wt-%) in THF (153 g) was added, which causes a slight temperature rise to 29° C. The mixture was stirred at 25° C. for 3 h. A HPLC assay demonstrated the formation of 5-chloro-3-methyl-2-(phenoxycarbonylamino)benzoic acid (intermediate) relative to the starting material in an area-% ratio of 5.2:1 at this stage. The final product could not yet be detected. After 22.5 h at 25° C. the reaction mixture was warmed up to 50° C. for additional 2 h. A HPLC assay demonstrated the formation of the product relative to the intermediate in an area-% ratio of 1:17.9 at this stage. The starting material was no longer detected. Then the reaction mixture was evaporated at a rotavap at 80° C./2 mbar. The solid residue was suspended in 300 g toluene and treated at reflux for 2 h. After that time a HPLC assay showed only traces of not converted intermediate. The suspension was cooled down to 5° C. The solid product was filtered, washed with 100 g of cold toluene and dried in vacuum dryer at 50° C./20 mbar overnight. 41.3 g product with a purity of 95.6 wt-% (quant HPLC) were obtained, i.e. a yield of 92.3%.

### Example 5

### 6-chloro-8-methyl-1H-3,1-benzoxazine-2,4-dione

2-Amino-5-chloro-3-methyl benzoic acid (237.7 g, calc. 97.6 wt-%) was suspended in toluene (900 g). A small amount of phenyl chloroformate (10 g, calc. 98 wt-%) was added at 25° C. The reaction mixture heated to reflux and then the main portion of phenyl chloroformate (194.5 g, calc. 98 wt-%) was added drop wise over 1.5 h at reflux. After the addition the mixture was kept at reflux over 6 h and then cooled down to 5° C. The solid product was filtered, washed 3 times with 100 g of cold toluene and dried in vacuum dryer at 50° C./20 mbar overnight. 263 g product with a purity of 97.76 wt-% (quant HPLC) were obtained, i.e. a yield of 97.2%.

### Example 6

### 6-chloro-8-methyl-1H-3,1-benzoxazine-2,4-dione

2-Amino-5-chloro-3-methyl benzoic acid (237.7 g, calc. 98.9 wt-%) was suspended in b-butyl acetate (900 g). A small amount of phenyl chloroformate (10 g, calc. 100 wt-%) was added at 25° C. The reaction mixture heated to reflux and then the main portion of phenyl chloroformate (194.5 g, calc. 100 wt-%) was added drop wise over 1.5 h at reflux. After the addition the mixture was kept at reflux over 5 h and then cooled down to 25° C. The solid product was filtered, washed 3 times with 100 g of n-butyl acetate and dried in vacuum dryer at 50° C./20 mbar overnight. 263.6 g product with a purity of >99 wt-% (quant H PLC) were obtained, i.e. a yield of 98.3%.

A detailed description, how the compounds of formula (I) can be converted to the compounds of formula (A), (A-1), (A-2), (A-3), and necessary intermediates, can be found in WO2013/076092. Following the procedures given there, and analogous methods, the following compounds of formula (A-3) can be synthesized, which are special compounds of the formula (A):

(A-3)

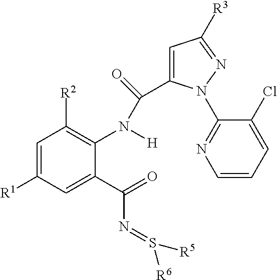

| Ex. A-3 | R⁵ | R⁶ | R² | R¹ | R³ |
|---|---|---|---|---|---|
| A-3-1 | C₂H₅ | C₂H₅ | Cl | Cl | CF₃ |
| A-3-2 | CH(CH₃)₂ | CH(CH₃)₂ | Cl | Cl | CF₃ |
| A-3-3 | CH₃ | CH₃ | Cl | Cl | CF₃ |
| A-3-4 | CH₂CH₂CH₂CH₂ | | Cl | Cl | CF₃ |
| A-3-5 | CH₃ | CH₃ | CH₃ | Cl | CF₃ |
| A-3-6 | C₂H₅ | C₂H₅ | CH₃ | Cl | CF₃ |
| A-3-7 | CH(CH₃)₂ | CH(CH₃)₂ | CH₃ | Cl | CF₃ |
| A-3-8 | CH₂CH₂CH₂CH₂ | | CH₃ | Cl | CF₃ |
| A-3-9 | C₂H₅ | C₂H₅ | Br | Cl | CF₃ |
| A-3-10 | CH(CH₃)₂ | CH(CH₃)₂ | Br | Cl | CF₃ |
| A-3-11 | C₂H₅ | C₂H₅ | Br | Br | CF₃ |
| A-3-12 | CH(CH₃)₂ | CH(CH₃)₂ | Br | Br | CF₃ |
| A-3-13 | C₂H₅ | C₂H₅ | CF₃ | Cl | CF₃ |
| A-3-14 | CH(CH₃)₂ | CH(CH₃)₂ | CF₃ | Cl | CF₃ |
| A-3-15 | C₂H₅ | C₂H₅ | CF₃ | Br | CF₃ |
| A-3-16 | CH(CH₃)₂ | CH(CH₃)₂ | CF₃ | Br | CF₃ |
| A-3-17 | C₂H₅ | C₂H₅ | Br | CF₃ | CF₃ |
| A-3-18 | CH(CH₃)₂ | CH(CH₃)₂ | Br | CF₃ | CF₃ |
| A-3-19 | C₂H₅ | C₂H₅ | Cl | CF₃ | CF₃ |
| A-3-20 | CH(CH₃)₂ | CH(CH₃)₂ | Cl | CF₃ | CF₃ |
| A-3-21 | C₂H₅ | C₂H₅ | Cl | CN | CF₃ |
| A-3-22 | CH(CH₃)₂ | CH(CH₃)₂ | Cl | CN | CF₃ |
| A-3-23 | C₂H₅ | C₂H₅ | CH₃ | CN | CF₃ |
| A-3-24 | CH(CH₃)₂ | CH(CH₃)₂ | CH₃ | CN | CF₃ |
| A-3-25 | CH₂CH₂CH₂CH₂ | | CH₃ | Cl | Br |
| A-3-26 | CH₃ | CH₃ | CH₃ | Cl | Br |
| A-3-27 | C₂H₅ | C₂H₅ | CH₃ | Cl | Br |
| A-3-28 | CH(CH₃)₂ | CH(CH₃)₂ | CH₃ | Cl | Br |
| A-3-29 | CH₂CH₂CH₂CH₂ | | Cl | Cl | Br |
| A-3-30 | CH₃ | CH₃ | Cl | Cl | Br |
| A-3-31 | C₂H₅ | C₂H₅ | Cl | Cl | Br |
| A-3-32 | CH(CH₃)₂ | CH(CH₃)₂ | Cl | Cl | Br |
| A-3-33 | CH₂CH₂CH₂CH₂ | | CH₃ | Cl | CHF₂ |
| A-3-34 | CH₃ | CH₃ | CH₃ | Cl | CHF₂ |
| A-3-35 | C₂H₅ | C₂H₅ | CH₃ | Cl | CHF₂ |
| A-3-36 | CH(CH₃)₂ | CH(CH₃)₂ | CH₃ | Cl | CHF₂ |
| A-3-37 | CH₂CH₂CH₂CH₂ | | Cl | Cl | CHF₂ |
| A-3-38 | CH₃ | CH₃ | Cl | Cl | CHF₂ |
| A-3-39 | C₂H₅ | C₂H₅ | Cl | Cl | CHF₂ |
| A-3-40 | CH(CH₃)₂ | CH(CH₃)₂ | Cl | Cl | CHF₂ |
| A-3-41 | C₂H₅ | C₂H₅ | Br | Cl | CHF₂ |
| A-3-42 | CH(CH₃)₂ | CH(CH₃)₂ | Br | Cl | CHF₂ |
| A-3-43 | C₂H₅ | C₂H₅ | Br | Br | CHF₂ |
| A-3-44 | CH(CH₃)₂ | CH(CH₃)₂ | Br | Br | CHF₂ |
| A-3-45 | C₂H₅ | C₂H₅ | CF₃ | Cl | CHF₂ |
| A-3-46 | CH(CH₃)₂ | CH(CH₃)₂ | CF₃ | Cl | CHF₂ |
| A-3-47 | C₂H₅ | C₂H₅ | CF₃ | Br | CHF₂ |
| A-3-48 | CH(CH₃)₂ | CH(CH₃)₂ | CF₃ | Br | CHF₂ |
| A-3-49 | C₂H₅ | C₂H₅ | Br | CF₃ | CHF₂ |
| A-3-50 | CH(CH₃)₂ | CH(CH₃)₂ | Br | CF₃ | CHF₂ |
| A-3-51 | C₂H₅ | C₂H₅ | Cl | CF₃ | CHF₂ |
| A-3-52 | CH(CH₃)₂ | CH(CH₃)₂ | Cl | CF₃ | CHF₂ |
| A-3-53 | C₂H₅ | C₂H₅ | Cl | CN | CHF₂ |
| A-3-54 | CH(CH₃)₂ | CH(CH₃)₂ | Cl | CN | CHF₂ |
| A-3-55 | C₂H₅ | C₂H₅ | CH₃ | CN | CHF₂ |
| A-3-56 | CH(CH₃)₂ | CH(CH₃)₂ | CH₃ | CN | CHF₂ |

For the details of the insecticidal properties of the compounds of formula (A-3), see e,g, WO2007/006670, WO2013/024009, and WO2013/024010.

Furthermore, by analogous methods, compounds of the formulas (A-1), (A-2), (A-4), (A-5) and (A-6) can be obtained.

(A-1)

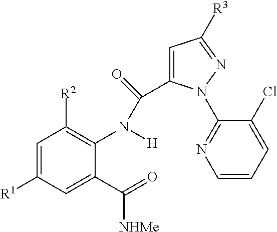

| Ex. A-1 | R² | R¹ | R³ |
|---|---|---|---|
| A-1-1 | CH₃ | Cl | CF₃ |
| A-1-2 | CH₃ | Cl | Br |
| A-1-3 | CH₃ | CN | CF₃ |
| A-1-4 | CH₃ | CN | Br |
| A-1-5 | Cl | CN | CF₃ |
| A-1-6 | Cl | CN | Br |
| A-1-7 | Cl | Cl | CF₃ |
| A-1-8 | Cl | Cl | Br |
| A-1-9 | Br | Cl | CF₃ |
| A-1-10 | Br | Cl | Br |
| A-1-11 | CH₃ | Cl | T * |
| A-1-12 | CH₃ | CN | T * |
| A-1-13 | Cl | CN | T * |
| A-1-14 | Br | Br | T * |
| A-1-15 | Br | Cl | T * |
| A-1-16 | Cl | Cl | T * |
| A-1-17 | Cl | Br | T * |

* T =

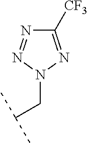

A particular embodiment is the process yielding A-1-2, which is known as chlorantraniliprole and is described in WO2003/015519.

A particular embodiment is the process yielding A-1-4, which is known as cyantraniliprole and is described in WO2004/067528.

A particular embodiment is the process yielding A-1-11, which is known as tetraniliprole and is described e.g. in WO2007144100, WO2010069502 or WO2011/157664.

(A-2)

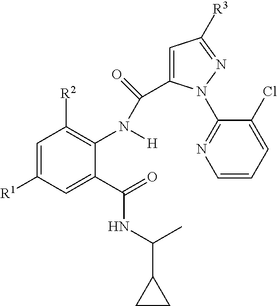

| Ex. A-2 | R² | R¹ | R³ |
|---|---|---|---|
| A-2-1 | Br | Cl | Br |
| A-2-2 | CH₃ | Cl | Br |
| A-2-3 | Br | Cl | CF₃ |
| A-2-4 | CH₃ | Cl | CF₃ |
| A-2-5 | Cl | CN | Br |
| A-2-6 | Cl | CN | Br |
| A-2-7 | Cl | CN | CF₃ |
| A-2-8 | Cl | CN | CF₃ |

A particular embodiment is the process yielding A-2-1, which is known as cyclaniliprole and is described in WO2005/077934.

(A-4)

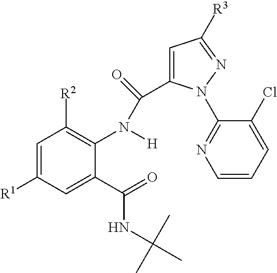

| Ex. A-4 | R² | R¹ | R³ |
|---|---|---|---|
| A-4-1 | CH₃ | Cl | CF₃ |
| A-4-2 | CH₃ | Cl | Br |
| A-4-3 | CH₃ | Cl | OCF₃ |
| A-4-4 | CH₃ | Cl | OCHF₂ |
| A-4-5 | CH₃ | Cl | OCH₂F |

A particular embodiment is the process yielding A-4-5, which is known as ZI-3757 and is described e.g. in WO2012/034403.

(A-5)

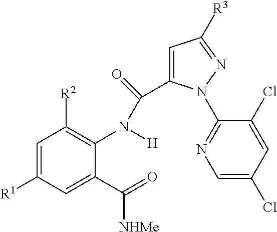

| Ex. A-5 | R² | R¹ | R³ |
|---|---|---|---|
| A-5-1 | CH₃ | Cl | CF₃ |
| A-5-2 | CH₃ | Cl | Br |
| A-5-3 | Cl | Cl | CF₃ |
| A-5-4 | Cl | Cl | Br |
| A-5-5 | CH₃ | Br | CF₃ |
| A-5-6 | CH₃ | Br | Br |
| A-5-7 | Br | Br | CF₃ |
| A-5-8 | Br | Br | Br |

A particular embodiment is the process yielding A-5-4, which is known as SYP-9080 and is described e.g. in US2011/046186.

(A-6)

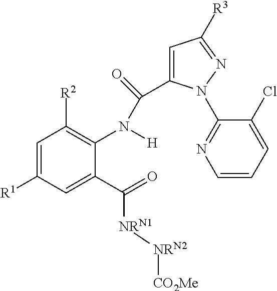

| Ex. A-6 | R² | R¹ | R³ | R^{N1} | R^{N2} |
|---|---|---|---|---|---|
| A-6-1 | CH₃ | Cl | CF₃ | H | H |
| A-6-2 | CH₃ | Cl | Br | H | H |
| A-6-3 | Cl | Cl | CF₃ | H | H |
| A-6-4 | Cl | Cl | Br | H | H |
| A-6-5 | CH₃ | Br | CF₃ | H | H |
| A-6-6 | CH₃ | Br | Br | H | H |
| A-6-7 | Br | Br | CF₃ | H | H |
| A-6-8 | Br | Br | Br | H | H |
| A-6-9 | CH₃ | Cl | CF₃ | H | CH₃ |
| A-6-10 | CH₃ | Cl | Br | H | CH₃ |
| A-6-11 | Cl | Cl | CF₃ | H | CH₃ |
| A-6-12 | Cl | Cl | Br | H | CH₃ |
| A-6-13 | CH₃ | Br | CF₃ | H | CH₃ |
| A-6-14 | CH₃ | Br | Br | H | CH₃ |
| A-6-15 | Br | Br | CF₃ | H | CH₃ |
| A-6-16 | Br | Br | Br | H | CH₃ |
| A-6-17 | CH₃ | Cl | CF₃ | CH₃ | H |
| A-6-18 | CH₃ | Cl | Br | CH₃ | H |
| A-6-19 | Cl | Cl | CF₃ | CH₃ | H |
| A-6-20 | Cl | Cl | Br | CH₃ | H |
| A-6-21 | CH₃ | Br | CF₃ | CH₃ | H |
| A-6-22 | CH₃ | Br | Br | CH₃ | H |
| A-6-23 | Br | Br | CF₃ | CH₃ | H |
| A-6-24 | Br | Br | Br | CH₃ | H |
| A-6-25 | CH₃ | Cl | CF₃ | CH₃ | CH₃ |
| A-6-26 | CH₃ | Cl | Br | CH₃ | CH₃ |
| A-6-27 | Cl | Cl | CF₃ | CH₃ | CH₃ |
| A-6-28 | Cl | Cl | Br | CH₃ | CH₃ |
| A-6-29 | CH₃ | Br | CF₃ | CH₃ | CH₃ |
| A-6-30 | CH₃ | Br | Br | CH₃ | CH₃ |
| A-6-31 | Br | Br | CF₃ | CH₃ | CH₃ |
| A-6-32 | Br | Br | Br | CH₃ | CH₃ |
| A-6-33 | CH₃ | Cl | CF₃ | CH₂CH₃ | H |
| A-6-34 | CH₃ | Cl | Br | CH₂CH₃ | H |
| A-6-35 | Cl | Cl | CF₃ | CH₂CH₃ | H |
| A-6-36 | Cl | Cl | Br | CH₂CH₃ | H |
| A-6-37 | CH₃ | Br | CF₃ | CH₂CH₃ | H |
| A-6-38 | CH₃ | Br | Br | CH₂CH₃ | H |
| A-6-39 | Br | Br | CF₃ | CH₂CH₃ | H |
| A-6-40 | Br | Br | Br | CH₂CH₃ | H |
| A-6-41 | CH₃ | Cl | CF₃ | H | CH₂CH₃ |
| A-6-42 | CH₃ | Cl | Br | H | CH₂CH₃ |
| A-6-43 | Cl | Cl | CF₃ | H | CH₂CH₃ |
| A-6-44 | Cl | Cl | Br | H | CH₂CH₃ |
| A-6-45 | CH₃ | Br | CF₃ | H | CH₂CH₃ |
| A-6-46 | CH₃ | Br | Br | H | CH₂CH₃ |
| A-6-47 | Br | Br | CF₃ | H | CH₂CH₃ |
| A-6-48 | Br | Br | Br | H | CH₂CH₃ |
| A-6-49 | CH₃ | Cl | CF₃ | CH₃ | CH₂CH₃ |
| A-6-50 | CH₃ | Cl | Br | CH₃ | CH₂CH₃ |
| A-6-51 | Cl | Cl | CF₃ | CH₃ | CH₂CH₃ |
| A-6-52 | Cl | Cl | Br | CH₃ | CH₂CH₃ |
| A-6-53 | CH₃ | Br | CF₃ | CH₃ | CH₂CH₃ |
| A-6-54 | CH₃ | Br | Br | CH₃ | CH₂CH₃ |
| A-6-55 | Br | Br | CF₃ | CH₃ | CH₂CH₃ |
| A-6-56 | Br | Br | Br | CH₃ | CH₂CH₃ |
| A-6-57 | CH₃ | Cl | CF₃ | CH₂CH₃ | CH₃ |
| A-6-58 | CH₃ | Cl | Br | CH₂CH₃ | CH₃ |
| A-6-59 | Cl | Cl | CF₃ | CH₂CH₃ | CH₃ |
| A-6-60 | Cl | Cl | Br | CH₂CH₃ | CH₃ |
| A-6-61 | CH₃ | Br | CF₃ | CH₂CH₃ | CH₃ |
| A-6-62 | CH₃ | Br | Br | CH₂CH₃ | CH₃ |
| A-6-63 | Br | Br | CF₃ | CH₂CH₃ | CH₃ |
| A-6-64 | Br | Br | Br | CH₂CH₃ | CH₃ |
| A-6-65 | CH₃ | Cl | CF₃ | CH₂CH₃ | CH₂CH₃ |
| A-6-66 | CH₃ | Cl | Br | CH₂CH₃ | CH₂CH₃ |
| A-6-67 | Cl | Cl | CF₃ | CH₂CH₃ | CH₂CH₃ |
| A-6-68 | Cl | Cl | Br | CH₂CH₃ | CH₂CH₃ |
| A-6-69 | CH₃ | Br | CF₃ | CH₂CH₃ | CH₂CH₃ |
| A-6-70 | CH₃ | Br | Br | CH₂CH₃ | CH₂CH₃ |
| A-6-71 | Br | Br | CF₃ | CH₂CH₃ | CH₂CH₃ |
| A-6-72 | Br | Br | Br | CH₂CH₃ | CH₂CH₃ |

The compounds of formula A-6 and the mentioned examples are described e.g. in WO2007/043677 or can be obtained and characterized in analogy thereto.

1.-15. (canceled)

16. A process for preparing a substituted isatoic acid anhydride compound of the formula (I) 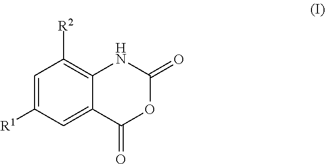
in which
R¹ is Cl, Br, I, or CN; and
R² is CH₃, Cl, Br;
comprising the step (b) of reacting a compound of the formula (II) 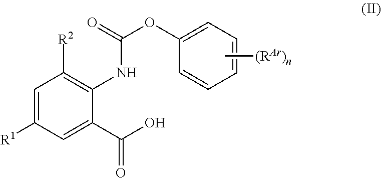
wherein R¹ and R² are as defined above; and
R^{Aa} is CH₃, Cl, NO₂ and n is 0, 1, 2, 3, 4 or 5;
by heating without any further reactants.

17. A process for preparing a compound of formula (II) 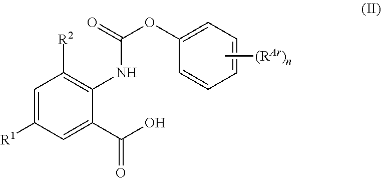
in which
R¹ is Cl, Br, I, or CN; and
R² is CH₃, Cl, Br;
R^{Ar} is CH₃, Cl, NO₂ and n is 0, 1, 2, 3, 4 or 5;
by reacting in a step (a) anthranilic acid derivative compounds of formula (III) 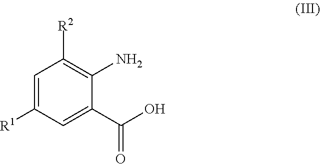
in which R¹ and R² are as defined above,
with a chloroformate compound of formula (IV) 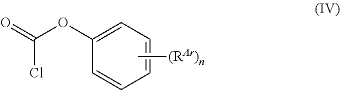
wherein R^{Ar} and n are as defined above;
in the presence of a solvent and without any further reactants.

18. The process according to claim 16, wherein the compound of formula (II) 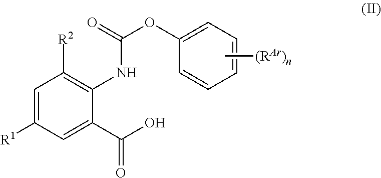
in which
R¹ is Cl, Br, I, or CN; and
R² is CH₃, Cl, Br; and
R^{Ar} is CH₃, Cl, NO₂ and n is 0, 1, 2, 3, 4 or 5;
is prepared in a step (a) by reacting an anthranilic acid derivative compound of formula (III) 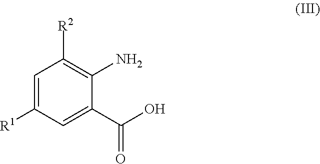
in which R¹ and R² are as defined above,
with a chloroformate compound of formula (IV) 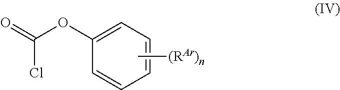
in which R^{Ar} and n are as defined above;
in the presence of a solvent and without any further reactants.

19. A process for preparing a substituted isatoic acid anhydride compound of the formula (I) 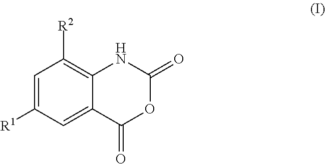
in which
R¹ is Cl, Br, I, or CN; and
R² is CH₃, Cl, Br;
wherein an anthranilic acid derivative compound of formula (III) 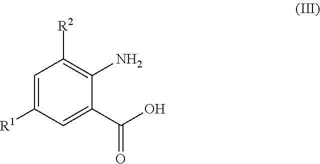
in which R¹ and R² are as defined above,
is reacted with a chloroformate compound of formula (IV) 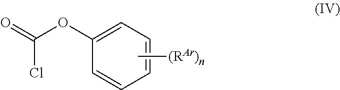
in which R^{Ar} and n are as defined above;
in the presence of a solvent and without any further reactants.

20. The process according to claim 16, in which
R¹ is Cl; and
R² is CH₃.

21. The process according to claim 16, in which the solvent is selected from aromatic hydrocarbon solvents or polar aprotic solvents.

22. The process according to claim 16, in which the solvent is selected from toluene, ethylbenzene, o-xylene, m-xylene, p-xylene, chlorbenzene, or a mixture thereof, preferably toluene.

23. The process according to claim 16, in which the solvent is selected from acetonitrile, n-butyl acetate and tetrahydrofurane.

24. The process according to claim 16, in which the reaction of step (b) is carried out at a temperature between 60 and 120° C.

25. The compound of formula (II) 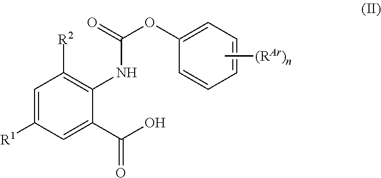
in which
R¹ is Cl, Br, I, or CN; and
R² is CH₃, Cl, Br; and
R^{Ar} is CH₃, Cl, NO₂ and n is 0, 1, 2, 3, 4 or 5;
with the proviso that the substituents R¹ and R² are not both Cl or not both Br at the same time.

26. The compound of formula (II) according to claim 25, in which
R¹ is Cl, Br, I, or CN; and
R² is CH₃, and
R^{Ar} is CH₃, Cl, NO₂ and n is 0, 1, 2, 3, 4 or 5.

27. The compound of formula (II-1a), which is a compound of formula (II) according to claim 25, in which R¹ is Cl and R² is CH₃ and n is 0:

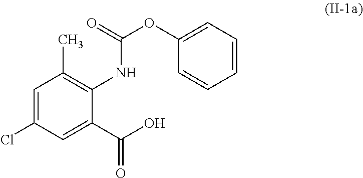 [SMILES: Cc1cc(Cl)cc(C(=O)O)c1NC(=O)Oc1ccccc1]

28. A method for preparing the compound of formula (A): 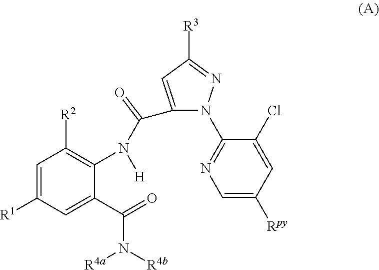
in which
R¹ is Cl, Br, I, or CN;
R² is CH₃, Cl, Br;
R³ is Cl, Br, I, CN, CF₃, CHF₂; OCH₂F or a residue of formula T: 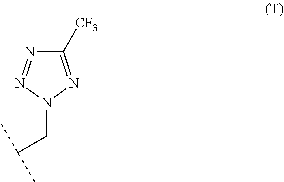
R^{py} is H or Cl;
R^{4a} and R^{4b} are independently selected from hydrogen, C₁-C₄-alkyl,
or
R^{4a} and R^{4b} together form a group (L) 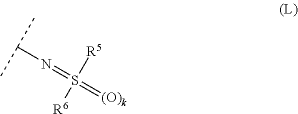
wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
and
R^{a} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄ alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy, (C₁-C₆-alkoxy)carbonyl, C₁-C₆-alkylamino and di-(C₁-C₆-alkyl)amino,
R^{b} is selected from the group consisting of C₁-C₆-alkyl, C₂-C₆-alkenyl, C₂-C₆-alkinyl, C₃-C₈-cycloalkyl, C₁-C₆-alkoxy, C₁-C₆-alkylthio, C₁-C₆-alkylsulfinyl, C₁-C₆-alkylsulfonyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 substituents selected from C₁-C₄-alkoxy; phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆-haloalkoxy and (C₁-C₆-alkoxy)carbonyl;
R^{c}, R^{d} are, independently from one another and independently of each occurrence, selected from the group consisting of hydrogen, cyano, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄-alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, phenyl, benzyl, pyridyl and phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy and (C₁-C₆-alkoxy)carbonyl; or R^{c} and R^{d}, together with the nitrogen atom to which they are bound, may form a 3-, 4-, 5-, 6- or 7-membered saturated, partially unsaturated or fully unsaturated heterocyclic ring which may additionally contain 1 or 2 further heteroatoms or heteroatom groups selected from N, O, S, NO, SO and SO₂, as ring members, where the heterocyclic ring may optionally be substituted with halogen, C₁-C₄-haloalkyl, C₁-C₄-alkoxy or C₁-C₄-haloalkoxy;
R^{e} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, —C(═NR^{c})NR^{c}R^{d}, phenyl, benzyl, pyridyl and phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy and C₁-C₆-haloalkoxy; or
two vicinal radicals R^{e} together form a group ═O, ═CH(C₁-C₄-alkyl), ═C(C₁-C₄-alkyl)C₁-C₄-alkyl, ═N(C₁-C₆-alkyl) or ═NO(C₁-C₆-alkyl).
R^{f} is independently selected from the group consisting of halogen, cyano, nitro, —OH, —SH, —SCN, C₁-C₆-alkyl, C₂-C₆-alkenyl, C₂-C₆-alkinyl, C₃-C₈-cycloalkyl, wherein one or more CH₂ groups of the aforementioned radicals may be replaced by a C═O group, and/or the aliphatic and cycloaliphatic moieties of the aforementioned radicals may be unsubstituted, partially or fully halogenated and/or may carry 1 or 2 radicals selected from C₁-C₄ alkoxy;
C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, —OR^{a}, —NR^{c}R^{d}, —S(O)ₙR^{a}, —S(O)ₙNR^{c}R^{d}, —C(═O)R^{a}, —C(═O)NR^{c}R^{d}, —C(═O)OR^{b}, —C(═S)R^{a}, —C(═S)NR^{c}R^{d}, —C(═S)OR^{b}, —C(═S)SR^{b}, —C(═NR^{c})R^{b}, and —C(═NR^{c})NR^{c}R^{d};
n is 0, 1 or 2;
comprising reacting a compound of formula (III) 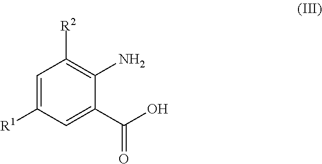
with a chloroformate compound of formula (IV) 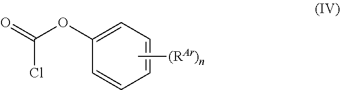
in the presence of a solvent and without any further reactants;
to obtain a compound of formula (II) 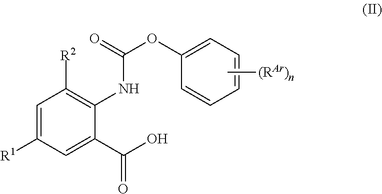
in which
R^{Ar} is CH₃, Cl, NO₂ and n is 0, 1, 2, 3, 4 or 5;
converting the compound of formula (II) to the compound of formula (A).

29. A process for preparing an anthranilamide compound of formula (A): 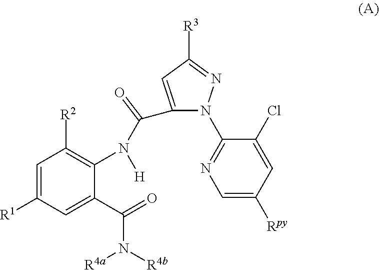
in which
R¹ is Cl, Br, I, or CN;
R² is CH₃, Cl, Br;
R³ is Cl, Br, I, CN, CF₃, CHF₂; OCH₂F or a residue of formula T: 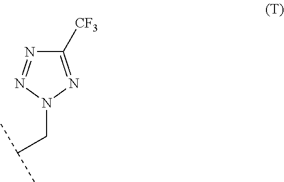
R^{py} is H or Cl;
R^{4a} and R^{4b} are independently selected from hydrogen, C₁-C₄-alkyl, C₃-C₈-cycloalkyl-C₁-C₄-alkyl, NR^{N2}—CO₂—C₁-C₄-alkyl, wherein R^{N2} is hydrogen, methyl or ethyl
or
R^{4a} and R^{4b} together form a group (L) 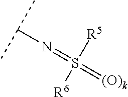
wherein
R⁵, R⁶ are selected independently of one another from the group consisting of hydrogen, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R^{e}, and phenyl, which is unsubstituted or carries 1 to 5 substituents R^{f}; or
R⁵ and R⁶ together represent a C₂-C₇-alkylene, C₂-C₇-alkenylene or C₆-C₉-alkynylene chain forming together with the sulfur atom to which they are attached a 3-, 4-, 5-, 6-, 7-, 8-, 9- or 10-membered saturated, partially unsaturated or fully unsaturated ring, wherein 1 to 4 of the CH₂ groups in the C₂-C₇-alkylene chain or 1 to 4 of any of the CH₂ or CH groups in the C₂-C₇-alkenylene chain or 1 to 4 of any of the CH₂ groups in the C₆-C₉-alkynylene chain may be replaced by 1 to 4 groups independently selected from the group consisting of C═O, C═S, O, S, N, NO, SO, SO₂ and NH, and wherein the carbon and/or nitrogen atoms in the C₂-C₇-alkylene, C₂-C₇-alkenylene or C₆-C₉-alkynylene chain may be substituted with 1 to 5 substituents independently selected from the group consisting of halogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl and C₂-C₆-haloalkynyl; said substituents being identical or different from one another if more than one substituent is present;
k is 0 or 1;
and
and wherein R^{a}, R^{b}, R^{c}, R^{d}, R^{e}, R^{f} and n are as defined in claim 28;
or a stereoisomer, salt, tautomer or N-oxide, or a polymorphic crystalline form, a co-crystal or a solvate of a compound or a stereoisomer, salt, tautomer or N-oxide thereof;
comprising heating a compound of the formula (II) without any further reactants 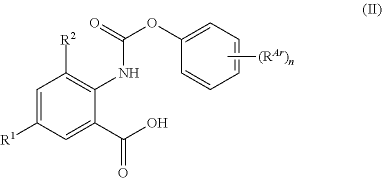
wherein R¹ and R² are as defined above; and
R^{Ar} is CH₃, Cl, NO₂ and n is 0, 1, 2, 3, 4 or 5;
to obtain a compound of the formula (I); 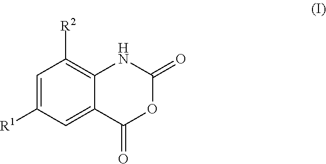
converting the compound of formula (I) to the compound of formula A).

30. The process according to claim 29, wherein in the compound of formula (A)
R¹ is Cl, Br, CN;
R² is CH₃, Cl, Br;
R³ is Br, CF₃; OCH₂F or a residue of formula T: 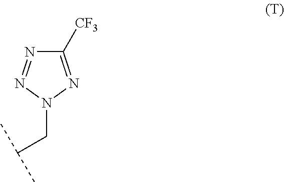
R^{py} is H or Cl;
R^{4a} and R^{4b} are one hydrogen and the other methyl, or
R^{4a} and R^{4b} are one hydrogen and the other cyclopropylmethyl, or
R^{4a} and R^{4b} are one hydrogen and the other cyclopropylethyl, or
R^{4a} and R^{4b} are one hydrogen and the other tert-butyl, or
R^{4a} and R^{4b} are one hydrogen and the other NR^{N2}—CO₂—CH₃, wherein R^{N2} is hydrogen, methyl or ethyl, or
R^{4a} and R^{4b} are one methyl and the other NR^{N2}—CO₂—CH₃, wherein R^{N2} is hydrogen, methyl or ethyl, or
R^{4a} and R^{4b} are one ethyl and the other NR^{N2}—CO₂—CH₃, wherein R^{N2} is hydrogen, methyl or ethyl, or
R^{4a} and R^{4b} together form a group (L) 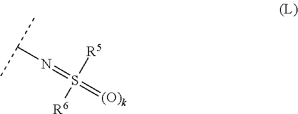
in which R⁵ and R⁶ are identical and selected from methyl, ethyl, isopropyl; and
k is 0.

31. The process according to claim 28, wherein in the compound of formula (A)
R¹ is Cl, Br, CN;
R² is CH₃, Cl, Br;
R³ is Br, CF₃; OCH₂F or a residue of formula T: 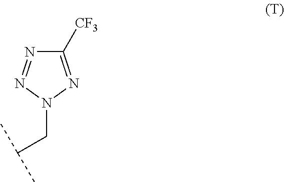
R^{py} is H or Cl;
R^{4a} and R^{4b} are one hydrogen and the other methyl, or
R^{4a} and R^{4b} are one hydrogen and the other cyclopropylmethyl, or
R^{4a} and R^{4b} are one hydrogen and the other cyclopropylethyl, or
R^{4a} and R^{4b} are one hydrogen and the other tert-butyl, or
R^{4a} a and R^{4b} are one hydrogen and the other NR^{N2}—CO₂—CH₃, wherein R^{N2} is hydrogen, methyl or ethyl, or
R^{4a} and R^{4b} are one methyl and the other NR^{N2}—CO₂—CH₃, wherein R^{N2} is hydrogen, methyl or ethyl, or
R^{4a} and R^{4b} are one ethyl and the other NR^{N2}—CO₂—CH₃, wherein R^{N2} is hydrogen, methyl or ethyl, or
R^{4a} and R^{4b} together form a group (L) 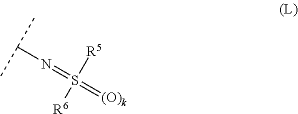
in which R⁵ and R⁶ are identical and selected from methyl, ethyl, isopropyl; and
k is 0.
